(19) United States
(12) Patent Application Publication
(10) Pub. No.: US 2026/0290521 A1
(43) Pub. Date: Sep. 24, 2026

(54) Recognition-Optimized Drug Discovery Platform: Computational Methods and Systems for Enhanced Identification and Prediction of Novel Drug Candidates Using Recognition Physics Principles

(71) Applicant: Jonathan Washburn, Austin, TX (US)

(72) Inventor: Jonathan Washburn, Austin, TX (US)

(21) Appl. No.: 19/570,777

(22) Filed: Mar. 18, 2026

Related U.S. Application Data
(60) Provisional application No. 63/774,200, filed on Mar. 19, 2025.

(51) Int. Cl. G16C 20/50

(52) U.S. Cl. CPC G16C 20/50

(57) ABSTRACT

A recognition-optimized drug discovery platform is disclosed for computationally ranking, filtering, selecting, and optimizing candidate compounds for one or more biological targets. The platform receives molecular, structural, descriptor-based, developability-related, and/or target-related inputs and transforms one or more such inputs into one or more nonnegative dimensionless recognition metrics. The recognition metrics are processed using a bounded recognition coverage function F_cov(r)=r/(r+X_opt), where r is a nonnegative dimensionless recognition metric and X_opt is a constant, to generate bounded recognition-weight values. The platform combines one or more bounded recognition-weight values with one or more transformed scoring values to generate recognition-weighted outputs for use in compound ranking, selection, deprioritization, or optimization. The platform can be implemented using one or more processors and one or more non-transitory computer-readable media in local, remote, distributed, or cloud-based computing environments.

### CROSS-REFERENCE TO RELATED APPLICATIONS

This application claims the benefit of U.S. Provisional Patent Application No. 63/774,200, filed Mar. 19, 2025, the disclosure of which is incorporated herein by reference in its entirety.

### BACKGROUND

The present disclosure relates generally to computational drug discovery, molecular modeling, predictive analytics, and computer-implemented systems for identifying, evaluating, ranking, and optimizing candidate therapeutic compounds. More particularly, the present disclosure relates to methods and systems that apply recognition-weighted computational processing to molecular docking, quantitative structure-activity relationship modeling, structural optimization, absorption-distribution-metabolism-excretion-toxicity evaluation, and virtual screening workflows in order to improve identification and prioritization of candidate drug compounds.

Computational drug discovery has become a central tool in pharmaceutical research and development. Existing drug discovery pipelines commonly employ molecular docking to estimate binding affinity between a candidate compound and a biological target, quantitative structure-activity relationship models to estimate likely biological activity from molecular descriptors, structural optimization methods to evaluate conformational and interaction-dependent properties, and absorption-distribution-metabolism-excretion-toxicity analysis to assess drug-likeness and developability. These techniques are frequently used in sequence or in parallel to triage large compound libraries and to prioritize compounds for synthesis, assay testing, and lead optimization. Although such tools have improved throughput and reduced experimental burden, conventional computational drug discovery approaches remain limited in several important respects. Molecular docking methods can assign high scores to compounds that adopt implausible poses or that lack meaningful structural correspondence to known bioactive ligands, thereby producing false positives. Conventional scoring functions generally do not provide a principled and bounded mechanism for discounting apparently favorable docking results when structural recognition is weak or absent. Quantitative structure-activity relationship approaches likewise exhibit shortcomings. Many such approaches treat descriptor magnitudes at face value, even where particular descriptor values are extreme, poorly calibrated, outside ordinary medicinal chemistry regimes, or otherwise non-representative of the patterns that most reliably correspond to true biological activity. As a result, individual descriptors or learned features can dominate predictions in a manner that reduces robustness and interpretability.

Structural optimization and conformational analysis methods also have limitations. Existing methods often emphasize local energetic terms without sufficiently accounting for recognition-weighted structural relationships across multiple interaction scales. In many discovery settings, such limitations can reduce the usefulness of conformational ranking, fragment-linking analysis, and structure-guided optimization.

Drug-likeness and developability assessment remain similarly fragmented. Conventional absorption-distribution-metabolism-excretion-toxicity tools often output multiple separate scores, warnings, or categorical indicators that must be manually reviewed and informally combined by a medicinal chemist or project team. This fragmented approach can make it difficult to generate a unified, bounded, and computationally consistent score for prioritization across large libraries.

Virtual screening workflows further suffer from integration problems. Even where docking, predictive modeling, and developability assessments are each individually available, existing systems often combine such outputs through ad hoc weighting, manual review, or empirically tuned scoring pipelines that are difficult to generalize across targets, target classes, and discovery campaigns. A need therefore exists for improved methods and systems that can transform heterogeneous drug discovery inputs into bounded, monotonic, and computationally coherent recognition-weighted outputs for ranking, filtering, prioritizing, selecting, and optimizing candidate compounds.

In some embodiments described herein, a recognition framework is used in which a nonnegative dimensionless recognition metric is processed according to a bounded recognition coverage function. In certain embodiments, the recognition coverage function is given by F_cov(r)=r/(r+X_opt), where r is a nonnegative dimensionless recognition metric and X_opt is a constant. In particular embodiments, X_opt=phi/pi. By applying this recognition coverage function across multiple computational drug discovery subsystems, the disclosed platform can provide a unified recognition-weighted framework for molecular matching, predictive modeling, structural optimization, developability scoring, and composite ranking.

A need accordingly remains for computer-implemented drug discovery systems capable of using a shared recognition-weighted computational framework across multiple drug discovery operations, including but not limited to docking-based evaluation, descriptor-based prediction, conformational or interaction-based optimization, developability assessment, and compound ranking within virtual screening workflows.

### SUMMARY

In one aspect, the present disclosure provides a computer-implemented method for evaluating candidate compounds using one or more recognition-weighted computational processes. The method can include receiving one or more inputs associated with a candidate compound, a biological target, or both, deriving one or more nonnegative dimensionless recognition metrics from the received inputs, applying a recognition coverage function to the one or more recognition metrics, and generating one or more recognition-weighted outputs used to rank, filter, select, deprioritize, or optimize candidate compounds.

In some embodiments, the recognition coverage function is F_cov(r)=r/(r+X_opt), where r is a nonnegative dimensionless recognition metric and X_opt is a constant. In certain embodiments, X_opt=phi/pi. In some embodiments, the same recognition coverage function is used across multiple subsystems of a computational drug discovery platform.

In one aspect, the present disclosure provides a recognition molecular matching subsystem configured to generate a recognition-weighted molecular match score for a candidate compound relative to a biological target. In some embodiments, the subsystem uses a docking-derived score together with a structural similarity metric associated with a known bioactive ligand, reference ligand, pharmacophore, interaction pattern, or other target-relevant reference representation.

In another aspect, the present disclosure provides a recognition-enhanced predictive modeling subsystem configured to generate activity-related predictions using molecular descriptors, learned features, latent variables, or combinations thereof. In some embodiments, descriptor values or other predictive inputs are weighted using the recognition coverage function in order to reduce disproportionate influence from extreme, weakly informative, or poorly calibrated values.

In another aspect, the present disclosure provides a recognition structural optimization subsystem configured to evaluate structural, conformational, energetic, or interaction-based properties of candidate compounds using recognition-weighted processing. In some embodiments, pairwise interaction values, distance-derived values, or related structural terms are weighted using the recognition coverage function to produce a recognition-weighted structural optimization output.

In another aspect, the present disclosure provides a recognition developability subsystem configured to generate a bounded drug-likeness or developability output from absorption-distribution-metabolism-excretion-toxicity-related metrics, physicochemical properties, safety-related metrics, or combinations thereof. In some embodiments, multiple such metrics are transformed into one or more normalized inputs and processed to produce a recognition-weighted developability score.

In another aspect, the present disclosure provides a recognition virtual screening ranking subsystem configured to combine outputs from two or more recognition-weighted subsystems into a composite prioritization output for a plurality of candidate compounds. In some embodiments, docking-related outputs, predictive outputs, and developability outputs are combined into a composite score used to rank compounds within a library or screening campaign.

In some embodiments, the disclosed methods and systems are implemented using one or more processors, one or more memory devices, and executable instructions stored in a non-transitory computer-readable medium. In some embodiments, the disclosed platform is implemented on local compute infrastructure, distributed compute infrastructure, accelerator hardware, cloud infrastructure, or combinations thereof.

In some embodiments, the disclosed platform is applicable to small-molecule discovery, fragment-based discovery, lead optimization, target-selective discovery, protein-protein interaction modulation, nucleic-acid targeting, multi-target screening, biologics-related payload prioritization, and other computational drug discovery contexts.

Additional features, embodiments, and advantages of the present disclosure will be apparent from the following description, drawings, and claims.

### BRIEF DESCRIPTION OF THE DRAWINGS

FIG. 1 is a schematic diagram of an example recognition-optimized drug discovery platform including computational subsystems for recognition molecular matching, recognition-enhanced predictive modeling, recognition structural optimization, recognition developability analysis, recognition virtual screening ranking, and associated computing and data-handling components.

FIG. 2 is a flow diagram of an example end-to-end recognition-weighted drug discovery workflow for receiving molecular and target-related inputs, preprocessing the inputs, generating one or more recognition metrics, applying recognition-weighted scoring, and producing prioritized candidate compound outputs.

FIG. 3 is a flow diagram of an example data ingestion and preprocessing workflow for receiving and processing compound library data, target structure data, reference ligand data, descriptor data, developability-related data, and associated computational inputs.

FIG. 4 is a flow diagram of an example recognition molecular matching workflow in which a candidate compound is evaluated relative to a biological target using docking-related information and one or more reference-associated similarity measures to generate a recognition-weighted molecular match score.

FIG. 5 is a diagram of an example shared recognition processing framework in which one or more inputs are transformed into one or more nonnegative dimensionless recognition metrics and processed using a recognition coverage function to generate bounded recognition-weighted outputs for use across multiple computational drug discovery subsystems.

FIG. 6 is a flow diagram of an example recognition-enhanced predictive modeling workflow in which molecular descriptors, learned features, latent variables, or combinations thereof are processed to generate one or more recognition-weighted activity-related predictions.

FIG. 7 is a flow diagram of an example recognition structural optimization workflow in which structural, conformational, interaction-based, or distance-derived values are processed using recognition-weighted evaluation to generate one or more structural optimization outputs.

FIG. 8 is a flow diagram of an example recognition developability analysis workflow in which physicochemical metrics, absorption-distribution-metabolism-excretion-toxicity-related metrics, safety-related metrics, or combinations thereof are transformed and processed to generate one or more recognition-weighted developability outputs.

FIG. 9 is a flow diagram of an example composite virtual screening ranking workflow in which outputs from two or more recognition-weighted subsystems are combined to generate a ranked, filtered, prioritized, or otherwise ordered set of candidate compounds.

FIG. 10 is a schematic diagram of an example multi-target and selectivity workflow in which recognition-weighted scoring is performed for a candidate compound relative to multiple targets in order to evaluate target preference, selectivity, off-target risk, or combinations thereof.

FIG. 11 is a flow diagram of an example feedback and model update workflow in which assay results, medicinal chemistry review, screening outcomes, or other campaign-related information are used to update one or more parameters, weights, references, thresholds, models, or workflow settings of the disclosed platform.

FIG. 12 is a schematic diagram of an example computing environment for implementing the disclosed platform using one or more processors, one or more memory devices, one or more data stores, one or more user interfaces, one or more communication interfaces, and one or more local, remote, distributed, cloud-based, or accelerator-supported computing resources.

### DETAILED DESCRIPTION

The following detailed description describes example embodiments of recognition-optimized computational drug discovery systems and methods. The disclosed embodiments are provided to illustrate principles of the invention and not to limit the scope of the claims. It will be understood that features described in connection with one embodiment may be used in combination with features of other embodiments unless otherwise indicated. It will also be understood that operations described as being performed by a particular subsystem can, in some embodiments, be performed by multiple subsystems, consolidated into fewer subsystems, reordered, repeated, omitted, or otherwise implemented in a different arrangement.

In the present disclosure, a candidate compound can include any chemical entity, molecular structure, fragment, scaffold, analog, derivative, conjugate component, prodrug, metabolite candidate, payload candidate, or other candidate therapeutic or diagnostic agent that can be computationally evaluated for interaction with a biological target or for suitability within a discovery workflow. In some embodiments, candidate compounds can include small molecules, fragment compounds, macrocycles, peptidomimetics, covalent warheads, linker-bearing compounds, nucleic-acid binders, protein-protein interaction modulators, or other chemical structures of interest.

A biological target can include any protein, nucleic acid, molecular complex, receptor, enzyme, ion channel, transporter, protein-protein interaction interface, nucleic-acid structure, cellular target, pathogen-associated target, or other target entity for which interaction with a candidate compound is to be evaluated. In some embodiments, the disclosed systems and methods can be used with kinases, G protein-coupled receptors, ion channels, proteases, nuclear receptors, epigenetic targets, transporters, protein-protein interaction targets, RNA targets, DNA-associated targets, viral targets, bacterial targets, fungal targets, inflammatory targets, oncology targets, or combinations thereof.

In some embodiments, the disclosed platform receives molecular, structural, descriptor-based, developability-related, experimental, predicted, or other computationally relevant inputs and converts one or more such inputs into one or more nonnegative dimensionless recognition metrics. In certain embodiments, the one or more recognition metrics are processed using a recognition coverage function to generate one or more bounded recognition-weighted outputs that can be used for ranking, filtering, selecting, deprioritizing, rescoring, clustering, or optimizing candidate compounds.

In some embodiments, the recognition coverage function is given by F_cov(r)=r/(r+X_opt), where r is a nonnegative dimensionless recognition metric and X_opt is a constant. In certain embodiments, X_opt=phi/pi. In some embodiments, the recognition coverage function produces a bounded monotonic output that increases as the recognition metric increases and that remains less than 1 for finite positive values of r. In certain embodiments, the recognition coverage function is used as a shared computational primitive across multiple subsystems of the disclosed platform.

As used herein, “phi” and “pi” refer to the mathematical constants commonly denoted by φ and π, respectively. In some embodiments, the constants are represented in plain ASCII text (e.g., “phi” and “pi”) to preserve compatibility across document formats.

In some embodiments, the disclosed platform applies the recognition coverage function to molecular matching operations, predictive modeling operations, structural optimization operations, developability analysis operations, composite ranking operations, selectivity operations, or combinations thereof. By using a shared recognition-weighted framework across multiple computational tasks, the platform can provide computational consistency across drug discovery stages that are otherwise evaluated using separate scoring systems, thereby enabling a unified approach to candidate prioritization.

FIG. 1 illustrates an example recognition-optimized drug discovery platform 100. In some embodiments, platform 100 includes one or more processors 102, memory 104, one or more data stores 106, and one or more communication interfaces 108. In some embodiments, platform 100 further includes a data ingestion and preprocessing subsystem 110, a recognition molecular matching subsystem 120, a recognition-enhanced predictive modeling subsystem 130, a recognition structural optimization subsystem 140, a recognition developability analysis subsystem 150, and a recognition virtual screening ranking subsystem 160. In some embodiments, platform 100 further includes a user interface and reporting subsystem 170, an external tool integration layer 180, and one or more external data sources 190.

In some embodiments, processors 102 execute instructions stored in memory 104 to perform one or more of the computational operations described herein. Memory 104 can include volatile memory, non-volatile memory, local memory, distributed memory, cache memory, or combinations thereof. Data stores 106 can store compound structure records, target structure records, reference ligand records, descriptor records, model parameters, scoring outputs, assay data, campaign data, workflow outputs, user settings, or other relevant computational information. Communication interfaces 108 can support communication between local and remote computing components, external software tools, remote databases, cloud resources, laboratory information systems, user-facing applications, or combinations thereof.

In some embodiments, data ingestion and preprocessing subsystem 110 receives one or more input data types including compound library data, target structure data, known ligand data, reference ligand data, descriptor data, developability-related data, assay-derived data, literature-derived data, or combinations thereof. In some embodiments, subsystem 110 standardizes, filters, reformats, augments, normalizes, or otherwise prepares received inputs before passing processed data to one or more downstream recognition-weighted subsystems.

In some embodiments, recognition molecular matching subsystem 120 receives information associated with a candidate compound and a biological target and generates a recognition-weighted molecular match score. In certain embodiments, subsystem 120 uses a docking-related score and one or more structural similarity measures associated with a reference ligand, known active compound, pharmacophore pattern, interaction profile, shape representation, electrostatic representation, or other target-relevant reference representation. In some embodiments, subsystem 120 is used to discount docking outcomes in circumstances where structural correspondence to known target-relevant recognition patterns is low.

In some embodiments, recognition-enhanced predictive modeling subsystem 130 receives descriptor values, learned features, latent variables, embeddings, or other predictive inputs associated with a candidate compound and generates one or more recognition-weighted activity-related predictions. In some embodiments, subsystem 130 applies recognition weighting to descriptor contributions, learned representations, grouped feature sets, latent dimensions, or other predictive inputs in order to reduce disproportionate sensitivity to poorly calibrated, outlier, weakly informative, or otherwise unstable inputs.

In some embodiments, recognition structural optimization subsystem 140 receives structural, conformational, energetic, geometric, or interaction-related information associated with a candidate compound and generates one or more recognition-weighted structural optimization outputs. In certain embodiments, subsystem 140 processes pairwise interaction values, distance-normalized values, conformational features, contact-derived values, atom-group interaction values, pharmacophore alignment values, or combinations thereof to produce outputs useful for conformer selection, structural prioritization, fragment linking, linker evaluation, macrocycle analysis, preorganization analysis, or related structure-guided tasks.

In some embodiments, recognition developability analysis subsystem 150 receives physicochemical metrics, developability metrics, safety-related metrics, absorption-distribution-metabolism-excretion-toxicity-related metrics, or combinations thereof and generates one or more recognition-weighted developability outputs. In some embodiments, subsystem 150 transforms multiple such inputs into normalized values and combines them to produce a bounded score useful for developability-based prioritization, filtering, triage, or project-stage gating.

In some embodiments, recognition virtual screening ranking subsystem 160 receives outputs from two or more of subsystems 120, 130, 140, and 150 and generates one or more composite prioritization outputs. In some embodiments, subsystem 160 produces a ranked list of candidate compounds for follow-up docking review, assay testing, medicinal chemistry analysis, focused library generation, lead optimization, target panel evaluation, or other downstream decision-making processes.

In some embodiments, user interface and reporting subsystem 170 presents ranked compound lists, score breakdowns, workflow status, model-related outputs, reference information, filter results, explanation outputs, or combinations thereof to a user. In some embodiments, subsystem 170 allows a user to configure workflow parameters, choose target structures, select reference ligands, define ranking thresholds, set weighting parameters, select output formats, or review recognition-weighted results generated by platform 100.

In some embodiments, external tool integration layer 180 enables platform 100 to interoperate with docking engines, cheminformatics software, descriptor-generation tools, molecular mechanics tools, machine learning frameworks, external databases, assay data systems, laboratory information systems, electronic laboratory notebooks, or other internal or external computational resources. In some embodiments, external data sources 190 include public databases, private compound libraries, internal assay repositories, structural repositories, medicinal chemistry data stores, project-specific target datasets, or combinations thereof.

In some embodiments, platform 100 can be implemented as a local workstation application, a server-based application, a cloud-based software service, a distributed workflow environment, an application programming interface-accessible platform, a plugin-enabled analysis framework, or any combination thereof. In some embodiments, one or more subsystems of platform 100 can execute on separate computing resources and exchange inputs and outputs over one or more network connections.

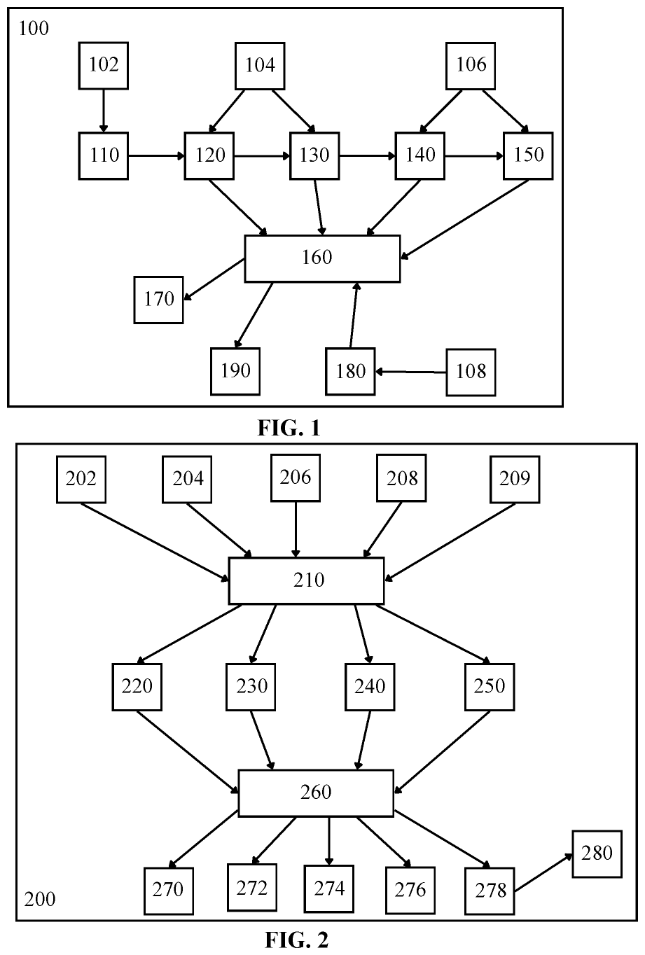

FIG. 2 illustrates an example end-to-end workflow 200 for generating recognition-weighted prioritization outputs. In some embodiments, workflow 200 begins with receipt of one or more candidate compound inputs 202, one or more biological target inputs 204, and one or more reference inputs 206. In some embodiments, additional inputs can include descriptor inputs 208, developability-related inputs 209, or one or more user-defined or campaign-defined workflow settings.

In some embodiments, the received inputs are preprocessed at step 210 to generate standardized molecular structures, prepared target structures, selected reference data, normalized descriptors, transformed developability inputs, generated conformers, or other processed inputs. In some embodiments, preprocessing step 210 includes one or more cleaning, normalization, validation, augmentation, deduplication, canonicalization, or format-conversion operations.

In some embodiments, one or more processed inputs are routed to a molecular matching operation 220, a predictive modeling operation 230, a structural optimization operation 240, a developability analysis operation 250, or combinations thereof. In some embodiments, one or more such operations generate intermediate values that are transformed into nonnegative dimensionless recognition metrics and processed using the recognition coverage function described herein.

In some embodiments, outputs from one or more operations 220, 230, 240, and 250 are combined at step 260 to generate a composite recognition-weighted prioritization output. In some embodiments, the composite output is used to produce a ranked candidate list 270, a filtered candidate set 272, a deprioritized candidate set 274, a selectivity assessment 276, a lead optimization worklist 278, a target panel report 280, or combinations thereof.

In some embodiments, workflow 200 is not limited to a single linear order. One or more operations can be omitted, repeated, reordered, parallelized, or conditionally invoked. For example, in some embodiments, predictive modeling can be performed before docking-related processing. In some embodiments, developability analysis can be used as an early filter before structural scoring. In some embodiments, structural optimization can be used to generate or select conformers prior to molecular matching. In some embodiments, composite ranking can be performed using outputs from two, three, four, or more subsystems depending on the needs of a given discovery campaign.

In some embodiments, workflow 200 can be used in hit identification, hit expansion, hit-to-lead progression, lead optimization, selectivity optimization, off-target screening, safety triage, fragment-based design, macrocycle prioritization, or other discovery workflows. In some embodiments, workflow 200 can also be used iteratively, such that outputs from one screening round are used to update reference inputs, thresholds, or weighting parameters for a subsequent screening round.

In some embodiments, the platform can apply the disclosed recognition-weighted framework to a full compound library, a focused subset of compounds, a project-defined chemical series, a medicinal chemistry worklist, an enumerated virtual library, or any combination thereof. In some embodiments, the platform can process candidate compounds individually, in batches, in streaming mode, or through distributed screening jobs executed across multiple compute resources.

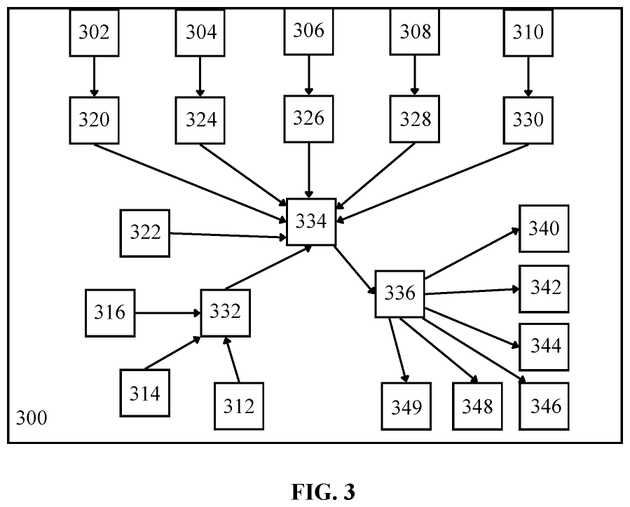

FIG. 3 illustrates an example data ingestion and preprocessing workflow 300. In some embodiments, workflow 300 begins with receipt of compound library data 302, target structure data 304, reference ligand data 306, descriptor-related data 308, and developability-related data 310. In some embodiments, one or more additional inputs can include assay-derived data 312, literature-derived data 314, campaign configuration data 316, or other computationally relevant information associated with a discovery workflow.

In some embodiments, compound library data 302 can include structure records expressed in one or more chemical formats including SMILES, SDF, MOL, MOL2, InChI, connection tables, graph-based representations, or other machine-readable compound formats. In some embodiments, compound library data 302 can be received from public databases, internal compound collections, virtual libraries, enumerated analog libraries, medicinal chemistry worklists, fragment libraries, focused screening collections, vendor catalogs, or combinations thereof.

In some embodiments, target structure data 304 can include experimentally determined structures, predicted structures, homology-based structures, consensus structures, or other target-associated structural representations. In some embodiments, target structure data 304 can include X-ray crystallography structures, cryo-electron microscopy structures, nuclear magnetic resonance structures, modeled structures, or predicted protein structures. In some embodiments, target structure data 304 can further include target annotations, binding site definitions, residue annotations, conformational-state labels, cofactor information, metal-ion information, or solvent-related information relevant to downstream computational analysis.

In some embodiments, reference ligand data 306 can include one or more known binders, co-crystallized ligands, known actives, known selective ligands, scaffold representatives, pharmacophore templates, interaction fingerprints, hot-spot models, shape models, electrostatic models, or other reference-associated data used to derive one or more recognition metrics. In some embodiments, reference ligand data 306 can be associated with one target, multiple targets, a target family, an off-target panel, or a project-defined selectivity objective.

In some embodiments, descriptor-related data 308 can include precomputed molecular descriptors, learned features, latent variables, embeddings, fingerprints, topological descriptors, physicochemical descriptors, graph-based descriptors, quantum-chemical descriptors, pharmacophore descriptors, or other feature data associated with one or more candidate compounds. In some embodiments, descriptor-related data 308 can be generated externally and imported into workflow 300, or can be generated internally by one or more components of platform 100.

In some embodiments, developability-related data 310 can include physicochemical properties, absorption-distribution-metabolism-excretion-toxicity-related metrics, safety-related metrics, permeability-related values, solubility-related values, clearance-related values, formulation-related values, aggregation-related values, or other properties relevant to drug-likeness, developability, or project-stage suitability. In some embodiments, developability-related data 310 can include experimentally measured values, predicted values, literature-derived values, inferred values, threshold flags, or combinations thereof.

In some embodiments, assay-derived data 312 can include potency measurements, binding measurements, selectivity measurements, functional readouts, toxicity results, permeability results, metabolism results, or other assay outputs associated with one or more candidate compounds or reference compounds. In some embodiments, literature-derived data 314 can include publications, curated activity records, public-database annotations, target-ligand associations, medicinal chemistry reports, or other textual or structured information used to enrich one or more downstream workflows.

In some embodiments, campaign configuration data 316 can include user-selected targets, project-stage settings, scoring preferences, threshold values, weighting parameters, normalization settings, workflow routing preferences, reference-selection settings, selectivity goals, or other configuration inputs that control one or more operations of workflow 300 or platform 100.

In some embodiments, compound library data 302 is processed at compound standardization step 320. In some embodiments, compound standardization step 320 includes one or more operations such as format conversion, valence correction, aromaticity normalization, tautomer handling, protonation-state assignment, stereochemistry normalization, salt stripping, solvent removal, duplicate detection, canonicalization, atom typing, bond-order normalization, or combinations thereof. In some embodiments, compound standardization step 320 produces standardized compound records suitable for descriptor generation, conformer generation, docking, similarity analysis, or downstream scoring.

In some embodiments, one or more standardized compound records are processed at conformer generation step 322. In some embodiments, conformer generation step 322 includes generation of two-dimensional-to-three-dimensional structures, force-field minimization, multiple-conformer enumeration, constrained conformer generation, macrocycle conformer generation, fragment conformer generation, stereoisomer expansion, or combinations thereof. In some embodiments, conformer generation step 322 produces one or more conformers for use in docking, molecular matching, structural optimization, pharmacophore comparison, or other downstream operations.

In some embodiments, target structure data 304 is processed at target preparation step 324. In some embodiments, target preparation step 324 includes chain selection, residue repair, side-chain completion, protonation-state assignment, loop modeling, binding-site definition, grid generation, solvation-state handling, cofactor retention or removal, metal treatment, pocket annotation, conformational-state selection, or combinations thereof. In some embodiments, target preparation step 324 produces one or more prepared target structures suitable for docking, interaction analysis, reference alignment, selectivity analysis, or other downstream target-associated operations.

In some embodiments, reference ligand data 306 is processed at reference selection and preparation step 326. In some embodiments, step 326 includes selection of one or more reference ligands based on known activity, co-crystal status, assay quality, structural diversity, selectivity profile, target-family relevance, pharmacophore relevance, interaction-pattern relevance, medicinal chemistry relevance, or combinations thereof. In some embodiments, step 326 can include preparation of one or more reference structures through standardization, conformer generation, pharmacophore extraction, interaction-fingerprint generation, shape-model generation, electrostatic-model generation, or combinations thereof.

In some embodiments, descriptor-related data 308 is processed at descriptor generation and preparation step 328. In some embodiments, step 328 includes descriptor calculation, feature selection, dimensionality reduction, latent-variable generation, embedding generation, normalization, missing-value handling, range correction, clipping, transformation, grouping, or combinations thereof. In some embodiments, descriptor generation and preparation step 328 produces one or more descriptor sets suitable for recognition-enhanced predictive modeling, clustering, filtering, or downstream prioritization.

In some embodiments, developability-related data 310 is processed at developability preprocessing step 330. In some embodiments, step 330 includes unit harmonization, directionality normalization, threshold transformation, target-window transformation, clipping, scaling, penalty conversion, missing-value handling, confidence tagging, or combinations thereof. In some embodiments, developability preprocessing step 330 produces one or more transformed developability inputs suitable for recognition-weighted developability analysis.

In some embodiments, assay-derived data 312 and literature-derived data 314 are processed at enrichment and annotation step 332. In some embodiments, step 332 includes matching compounds to assay records, assigning activity labels, associating compounds with target-specific annotations, importing selectivity records, importing mechanism-of-action annotations, associating literature-derived evidence, quality-filtering imported records, or combinations thereof. In some embodiments, enrichment and annotation step 332 produces annotated records that can support model training, reference selection, validation, threshold setting, or prioritization.

In some embodiments, one or more outputs of steps 320, 322, 324, 326, 328, 330, and 332 are processed at validation and quality-control step 334. In some embodiments, validation and quality-control step 334 includes syntax validation, record completeness checks, duplicate checks, structural sanity checks, descriptor sanity checks, target-preparation checks, missing-data checks, file-integrity checks, range checks, confidence scoring, or combinations thereof. In some embodiments, invalid or low-confidence records can be excluded, flagged, repaired, routed for manual review, or processed under a fallback workflow.

In some embodiments, one or more validated outputs are processed at normalization and transformation step 336 to generate one or more normalized or transformed inputs suitable for derivation of one or more nonnegative dimensionless recognition metrics. In some embodiments, step 336 includes affine scaling, min-max scaling, percentile transformation, z-score-related transformation, rank-based transformation, reciprocal transformation, exponential transformation, logarithmic transformation, clipping, offsetting, sign inversion, absolute-value conversion, threshold-based conversion, or combinations thereof.

In some embodiments, normalization and transformation step 336 is used to convert raw scores, raw descriptor values, raw distance values, raw energetic values, or raw developability values into one or more transformed values that are nonnegative and dimensionless or otherwise suitable for subsequent normalization into nonnegative dimensionless recognition metrics. In some embodiments, a transformed value can be divided by a selected scale, a reference magnitude, a percentile-derived value, a target-specific scale factor, or another normalization factor to produce a recognition metric r for use in the recognition coverage function.

In some embodiments, workflow 300 produces one or more processed compound inputs 340, one or more processed target inputs 342, one or more processed reference inputs 344, one or more processed descriptor inputs 346, one or more processed developability inputs 348, and one or more processed annotation or validation outputs 349. In some embodiments, one or more such outputs are routed to recognition molecular matching subsystem 120, recognition-enhanced predictive modeling subsystem 130, recognition structural optimization subsystem 140, recognition developability analysis subsystem 150, recognition virtual screening ranking subsystem 160, or combinations thereof.

In some embodiments, workflow 300 is modular and configurable. For example, in some embodiments, a discovery campaign can use precomputed descriptors while omitting internal descriptor generation. In some embodiments, a campaign can use a single co-crystallized ligand as a reference, while in other embodiments a campaign can use multiple known actives, a pharmacophore ensemble, or one or more interaction-pattern references. In some embodiments, a campaign can omit assay-derived enrichment if no internal assay data is available. In some embodiments, a campaign can use only docking and developability inputs, or only descriptor and selectivity inputs, depending on project objectives.

In some embodiments, workflow 300 can be executed once at the beginning of a screening campaign, periodically during campaign progression, continuously in response to newly received data, or iteratively after one or more rounds of assay review, medicinal chemistry review, or target-profile refinement. In some embodiments, one or more outputs of workflow 300 can be cached, versioned, logged, or stored in data stores 106 for later reuse in validation, auditing, retraining, selectivity analysis, or follow-up screening campaigns.

In some embodiments, preprocessing operations described with respect to FIG. 3 are not limited to the specific steps shown. One or more preprocessing operations can be omitted, merged, subdivided, repeated, reordered, or performed in parallel. In some embodiments, preprocessing can be performed locally, remotely, in a distributed computing environment, through one or more external software tools, or through combinations thereof.

In some embodiments, the outputs of workflow 300 provide the computational basis for subsequent derivation of one or more recognition metrics used throughout the disclosed platform. By standardizing and transforming heterogeneous compound, target, reference, descriptor, and developability inputs into forms suitable for recognition-weighted processing, workflow 300 enables consistent application of the shared recognition framework across multiple downstream subsystems.

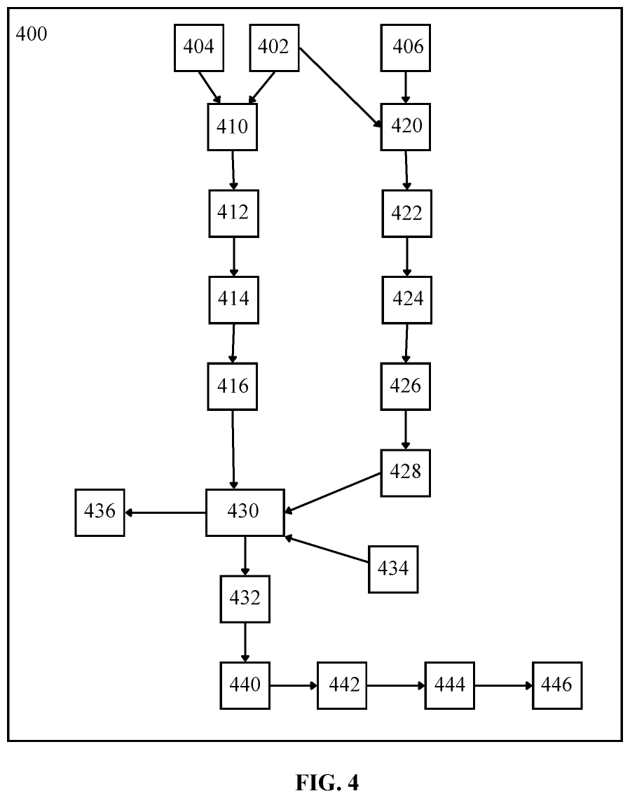

FIG. 4 illustrates an example recognition molecular matching workflow 400. In some embodiments, workflow 400 is used to generate a recognition-weighted molecular match output for a candidate compound relative to a biological target. In certain embodiments, workflow 400 combines a docking-related value with one or more reference-associated similarity measures in order to reduce the impact of apparently favorable docking outcomes that are not accompanied by sufficient target-relevant structural recognition.

In some embodiments, workflow 400 begins with receipt of one or more processed compound inputs 402, one or more processed target inputs 404, and one or more processed reference inputs 406. In some embodiments, processed compound inputs 402 can include one or more standardized structures, one or more generated conformers, one or more stereochemical forms, or combinations thereof. In some embodiments, processed target inputs 404 can include one or more prepared target structures, binding-site definitions, docking grids, residue annotations, or combinations thereof. In some embodiments, processed reference inputs 406 can include one or more reference ligands, co-crystallized ligands, known actives, pharmacophore representations, interaction fingerprints, shape models, electrostatic models, or other reference-associated target-relevant representations.

In some embodiments, processed compound inputs 402 and processed target inputs 404 are routed to docking-related evaluation step 410. In some embodiments, docking-related evaluation step 410 includes one or more rigid docking operations, flexible docking operations, induced-fit docking operations, constrained docking operations, ensemble docking operations, consensus docking operations, or combinations thereof. In some embodiments, docking-related evaluation step 410 produces one or more raw docking-related values 412 associated with one or more candidate compound poses relative to the biological target.

In some embodiments, the one or more raw docking-related values 412 can include docking energies, affinity estimates, interaction scores, pose-quality values, docking ranks, consensus docking values, force-field-derived estimates, or combinations thereof. In some embodiments, one or more such raw values can be signed, unbounded, model-specific, tool-specific, or otherwise not directly suitable for use as a nonnegative dimensionless recognition metric.

In some embodiments, one or more raw docking-related values 412 are processed at docking-value transformation step 414 to generate one or more transformed docking values 416. In some embodiments, docking-value transformation step 414 includes negation, offsetting, absolute-value conversion, clipping, rank-based conversion, percentile-based conversion, affine scaling, bounded monotonic transformation, or combinations thereof. In some embodiments, one or more transformed docking values 416 are selected so that larger values correspond to more favorable docking-related outcomes.

In some embodiments, processed compound inputs 402 and processed reference inputs 406 are also routed to similarity derivation step 420. In some embodiments, similarity derivation step 420 includes generation of one or more structural similarity values, shape similarity values, electrostatic similarity values, pharmacophore overlap values, interaction-fingerprint similarity values, graph similarity values, fingerprint similarity values, conformer-alignment similarity values, RMSD-derived values, or combinations thereof. In some embodiments, similarity derivation step 420 can be performed relative to a single reference input or relative to multiple reference inputs.

In some embodiments, similarity derivation step 420 can use one or more similarity or correspondence measures including Tanimoto similarity, Dice similarity, cosine similarity, overlap coefficients, graph-based similarity, maximum common substructure-derived correspondence, pharmacophore feature correspondence, shape-overlap scores, electrostatic overlap scores, atom-mapping-derived correspondence values, interaction-pattern overlap values, contact-map similarity values, or combinations thereof. In some embodiments, workflow 400 can additionally or alternatively use one or more distance-derived values that are transformed into similarity-like values for use in recognition processing.

In some embodiments, one or more raw similarity or correspondence values generated at step 420 are processed at recognition-metric derivation step 422 to generate one or more nonnegative dimensionless recognition metrics 424. In some embodiments, step 422 includes normalization relative to a selected scale, a reference value, a target-specific factor, a percentile-derived factor, a project-defined factor, or another normalization basis. In some embodiments, one or more raw values are transformed before normalization through clipping, offsetting, reciprocal conversion, exponential conversion, threshold conversion, or combinations thereof.

In some embodiments, the one or more nonnegative dimensionless recognition metrics 424 are processed at recognition coverage step 426 using the recognition coverage function F_cov(r)=r/(r+X_opt), where r is a nonnegative dimensionless recognition metric and X_opt is a constant. In certain embodiments, X_opt=phi/pi. In some embodiments, recognition coverage step 426 produces one or more bounded recognition-weight values 428 that increase with increasing recognition metric magnitude while remaining less than 1 for finite positive values of r.

In some embodiments, one or more transformed docking values 416 and one or more bounded recognition-weight values 428 are combined at molecular match output step 430 to generate one or more recognition-weighted molecular match outputs 432. In some embodiments, a recognition-weighted molecular match output can be expressed as R_match(C,T)=DockScore(C,T)*F_cov(D_C,T), where DockScore(C,T) is a transformed docking-related value associated with candidate compound C and target T, and D_C,T is a nonnegative dimensionless recognition metric associated with candidate compound C relative to one or more references relevant to target T.

In some embodiments, the value D_C,T can be derived from a single reference ligand. In some embodiments, D_C,T can be derived from multiple reference ligands, multiple reference conformers, multiple pharmacophore models, multiple interaction fingerprints, or combinations thereof. In some embodiments, D_C,T can be based on a maximum value, average value, weighted value, consensus value, median value, threshold-qualified value, or other derived value computed from multiple reference-associated measures.

In some embodiments, workflow 400 can include optional pose-selection step 434. In some embodiments, step 434 selects one or more candidate poses for use in molecular matching based on docking rank, interaction features, binding-site occupancy, geometric plausibility, contact quality, reference similarity, or combinations thereof. In some embodiments, recognition-weighted molecular match output 432 can be generated for a single selected pose, a set of poses, a best-scoring pose, a consensus pose representation, or multiple alternative poses.

In some embodiments, workflow 400 can include optional target-panel evaluation step 436 in which a candidate compound is evaluated relative to multiple targets using corresponding target-specific references. In some embodiments, target-panel evaluation step 436 can generate a set of recognition-weighted molecular match outputs 432 for use in selectivity analysis, off-target analysis, polypharmacology assessment, or target-prioritization workflows.

In some embodiments, workflow 400 can produce one or more ranked molecular match outputs 440, one or more filtering decisions 442, one or more deprioritization decisions 444, one or more selectivity-related outputs 446, or combinations thereof. In some embodiments, ranked molecular match outputs 440 can be used directly to prioritize compounds for further computational analysis or experimental testing. In some embodiments, filtering decisions 442 can exclude compounds having low recognition-weighted molecular match values despite favorable raw docking-related values. In some embodiments, deprioritization decisions 444 can identify compounds that exhibit insufficient reference-associated recognition relative to a project objective.

In some embodiments, workflow 400 can be used in hit identification, hit expansion, lead optimization, scaffold hopping, selectivity optimization, fragment-based discovery, covalent discovery, protein-protein interaction modulation, nucleic-acid targeting, or combinations thereof. In some embodiments, a reference representation used in workflow 400 can correspond to a known binder, a selective binder, a privileged scaffold, a co-crystallized ligand, a binding hot-spot representation, a nucleic-acid motif binder, or another target-relevant recognition template.

In some embodiments, the recognition weighting applied in workflow 400 provides a bounded attenuation mechanism rather than an unbounded amplification mechanism. In certain embodiments, the bounded recognition-weight value 428 does not increase a transformed docking value beyond the magnitude of the transformed docking value itself. In some embodiments, if the recognition metric is zero or substantially absent, the corresponding recognition-weighted molecular match output can be zero, substantially reduced, or otherwise suppressed relative to the transformed docking value.

In some embodiments, workflow 400 is configurable with respect to docking tool choice, transformation choice, similarity metric choice, reference selection strategy, threshold settings, pose-selection logic, or output routing. In some embodiments, one or more components of workflow 400 can be omitted, repeated, reordered, parallelized, or distributed across multiple computing resources. In some embodiments, workflow 400 can be executed as a stand-alone scoring module, as part of an integrated platform workflow, or in combination with predictive modeling, structural optimization, developability analysis, or composite ranking operations described elsewhere herein.

In some embodiments, docking-value transformation step 414 sets DockScore(C,T)=clip(−E_dock(C,T)+b, 0, S_max), where E_dock(C,T) is a docking energy in kcal/mol, b is an offset selected in a range of 0 to 20, and S_max is selected in a range of 10 to 50. In some embodiments, when a docking tool produces an affinity estimate or interaction score rather than an energy, step 414 applies an affine mapping followed by clipping such that the transformed docking value is nonnegative and bounded.

In some embodiments, similarity derivation step 420 produces a similarity Sim in [0,1] (for example, a Tanimoto similarity of fingerprints), and recognition-metric derivation step 422 sets r=clip(Sim/S_ref, 0, r_max), where S_ref is a normalizing scale. In some embodiments, S_ref is selected as (i) a percentile-derived scale computed from similarity values over a screening library for the target (for example, a 90th-99th percentile), (ii) a reference-derived scale computed from one or more reference ligands, or (iii) a fixed scale stored in memory. In some embodiments, r_max is selected in a range of 1 to 10 such that the recognition metric is nonnegative, dimensionless, and bounded.

In some embodiments, a compound is selected at ranking and filtering step 442 when R_match(C,T) satisfies a decision rule. In some embodiments, the decision rule uses a threshold θ such that the compound is advanced when R_match(C,T)≥θ, where θ is selected as (i) a percentile threshold over a screened library (for example, top 1%-10%) or (ii) a fixed threshold stored in memory. In some embodiments, the decision rule uses a top-K selection where K is selected in a range of 10 to 10,000 depending on library size. In some embodiments, the platform stores θ, K, S_ref, and X_opt with campaign metadata for later review or reanalysis.

In some embodiments, one or more outputs of workflow 400 are provided to recognition virtual screening ranking subsystem 160, user interface and reporting subsystem 170, feedback and model update workflows, or other downstream operations of platform 100. In some embodiments, one or more outputs of workflow 400 can also be stored, versioned, logged, or associated with campaign-specific metadata for later review, validation, or reanalysis.

By combining docking-related evaluation with bounded recognition weighting derived from one or more reference-associated recognition metrics, workflow 400 enables molecular matching decisions that are more sensitive to target-relevant structural correspondence than docking-related evaluation alone. In some embodiments, this can reduce false-positive prioritization of compounds that appear favorable under docking-related scoring but lack sufficient recognition similarity to target-relevant reference patterns.

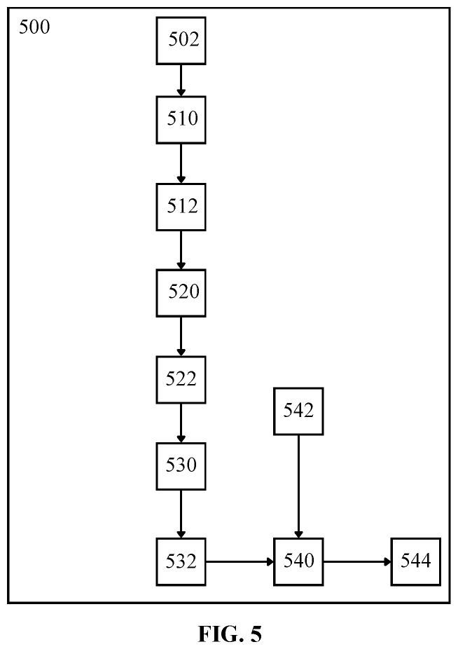

FIG. 5 illustrates an example shared recognition processing framework 500 that can be used across multiple computational drug discovery subsystems of platform 100. In some embodiments, framework 500 receives one or more raw inputs 502 and converts the one or more raw inputs into one or more nonnegative dimensionless recognition metrics suitable for processing by a recognition coverage function. In some embodiments, the resulting bounded recognition-weighted outputs are used in molecular matching, predictive modeling, structural optimization, developability analysis, composite ranking, selectivity analysis, or combinations thereof.

In some embodiments, raw inputs 502 can include raw docking-related values, raw similarity values, raw descriptor values, raw latent-variable values, raw interaction values, raw distance values, raw developability values, raw safety-related values, raw target-panel values, raw assay-derived values, or other computationally relevant values generated by one or more upstream operations. In some embodiments, raw inputs 502 can be obtained from internal modules of platform 100, external software tools, imported datasets, predicted models, experimentally derived records, or combinations thereof.

In some embodiments, one or more raw inputs 502 are processed at optional transformation step 510 to generate one or more transformed inputs 512. In some embodiments, transformation step 510 includes one or more negation operations, offsetting operations, clipping operations, absolute-value conversions, percentile-based transformations, rank-based transformations, reciprocal transformations, exponential transformations, logarithmic transformations, bounded monotonic transformations, target-window transformations, penalty-to-benefit conversions, or combinations thereof. In some embodiments, transformation step 510 is used to place one or more inputs into a form in which larger values correspond to greater target-relevant recognition, greater predictive utility, greater structural favorability, greater developability favorability, or other desired interpretation.

In some embodiments, transformed inputs 512 are processed at normalization step 520 to generate one or more nonnegative dimensionless recognition metrics 522. In some embodiments, normalization step 520 includes division by a selected scale, a reference magnitude, a target-specific scale factor, a project-specific scale factor, a percentile-derived normalization factor, a descriptor-family normalization factor, or another selected normalization basis. In some embodiments, normalization step 520 can additionally include clipping, floor enforcement, ceiling enforcement, thresholding, confidence weighting, grouping, or combinations thereof.

In some embodiments, a recognition metric r generated at normalization step 520 is nonnegative and dimensionless. In some embodiments, a recognition metric r can be derived from a transformed value divided by a selected scale. In some embodiments, a recognition metric r can be derived from a similarity-like value, a correspondence value, a normalized descriptor-related value, a normalized interaction-related value, a normalized developability-related value, or combinations thereof. In some embodiments, multiple recognition metrics can be generated from a single raw input stream or from multiple different raw input streams.

In some embodiments, one or more nonnegative dimensionless recognition metrics 522 are processed at recognition coverage step 530 using a recognition coverage function. In some embodiments, the recognition coverage function is F_cov(r)=r/(r+X_opt), where r is a nonnegative dimensionless recognition metric and X_opt is a constant. In certain embodiments, X_opt=phi/pi. In some embodiments, recognition coverage step 530 produces one or more bounded recognition-weight outputs 532.

In some embodiments, the bounded recognition-weight outputs 532 are monotonic with respect to the corresponding recognition metrics 522. In some embodiments, a larger recognition metric produces a larger bounded recognition-weight output. In some embodiments, a recognition metric of zero produces a bounded recognition-weight output of zero. In some embodiments, finite positive recognition metrics produce bounded recognition-weight outputs greater than zero and less than one. In some embodiments, such bounded behavior enables attenuation or weighting of downstream values without requiring unbounded amplification.

In some embodiments, one or more bounded recognition-weight outputs 532 are combined at output integration step 540 with one or more base computational values 542 to generate one or more recognition-weighted outputs 544. In some embodiments, a base computational value 542 can include a transformed docking-related value, a descriptor contribution value, a predictive feature contribution, an interaction-derived value, a developability-related value, a subsystem score, a target-specific score, or another computational value suitable for weighting or modulation.

In some embodiments, a recognition-weighted output 544 can be expressed in the general form S_rec=S_base*F_cov(r_rec), where S_base is a base computational value and r_rec is a corresponding nonnegative dimensionless recognition metric. In some embodiments, S_base can already be normalized, transformed, shifted, clipped, ranked, or otherwise prepared prior to combination with F_cov(r_rec). In some embodiments, one or more recognition-weighted outputs 544 can be combined, aggregated, ranked, filtered, or further transformed by downstream modules of platform 100.

In some embodiments, framework 500 can be applied to a molecular matching workflow in which a docking-related value is weighted by a recognition metric derived from reference-associated structural similarity. In some embodiments, framework 500 can be applied to a predictive modeling workflow in which one or more descriptor-related or latent-variable-related contributions are weighted by one or more corresponding recognition metrics. In some embodiments, framework 500 can be applied to a structural optimization workflow in which interaction-related or distance-derived values are weighted using one or more recognition metrics. In some embodiments, framework 500 can be applied to a developability workflow in which one or more transformed developability-related inputs are combined into a recognition-weighted developability output.

In some embodiments, framework 500 can additionally be applied to composite ranking, selectivity scoring, panel scoring, target-family scoring, off-target analysis, feedback-guided rescoring, or other higher-level platform operations. In some embodiments, the reuse of framework 500 across multiple computational subsystems provides a shared mathematical and computational basis for integrating otherwise heterogeneous drug discovery inputs.

In some embodiments, transformation step 510 and normalization step 520 are configurable on a per-input, per-module, per-target, per-project, or per-campaign basis. For example, in some embodiments, a docking-related value can be negated and offset before normalization, while in some embodiments a similarity-related value can be used directly or after clipping. In some embodiments, a developability-related value can be directionally transformed so that higher transformed values correspond to more favorable developability prior to normalization. In some embodiments, a descriptor-related input can be grouped or clipped before derivation of one or more recognition metrics.

In some embodiments, framework 500 can process a single recognition metric or multiple recognition metrics in parallel. In some embodiments, multiple recognition metrics can be processed independently and then combined. In some embodiments, multiple recognition metrics can be aggregated before application of the recognition coverage function. In some embodiments, separate recognition-weight outputs can be generated for separate features, feature groups, target-specific channels, structural channels, or subsystem-specific channels.

In some embodiments, framework 500 can be implemented using scalar processing, vectorized processing, matrix-based processing, tensor-based processing, batch processing, streaming processing, distributed processing, or combinations thereof. In some embodiments, one or more components of framework 500 can execute on one or more central processing units, graphics processing units, field-programmable gate arrays, tensor accelerators, remote servers, cloud resources, or combinations thereof.

In some embodiments, one or more values generated by framework 500 can be logged, cached, versioned, exported, displayed, or associated with one or more audit records. In some embodiments, the platform can store transformed inputs 512, recognition metrics 522, bounded recognition-weight outputs 532, base computational values 542, and recognition-weighted outputs 544 for later inspection, explanation generation, threshold tuning, model validation, campaign comparison, or follow-up screening.

In some embodiments, framework 500 is not limited to the exact sequence shown. One or more operations can be omitted, reordered, repeated, subdivided, merged, or conditionally applied. For example, in some embodiments, a raw input 502 can already be nonnegative and dimensionless, in which case transformation step 510 or normalization step 520 can be omitted or simplified. In some embodiments, one or more intermediate steps can include confidence adjustment, uncertainty weighting, threshold qualification, or project-specific calibration before or after recognition coverage step 530.

By providing a shared procedure for transforming heterogeneous computational inputs into nonnegative dimensionless recognition metrics and then converting such metrics into bounded recognition-weight outputs, framework 500 enables multiple modules of platform 100 to operate using a common recognition-weighted computational logic. In some embodiments, this supports consistent scoring behavior across molecular matching, predictive modeling, structural optimization, developability analysis, and composite ranking operations, thereby improving interoperability and interpretability across the platform.

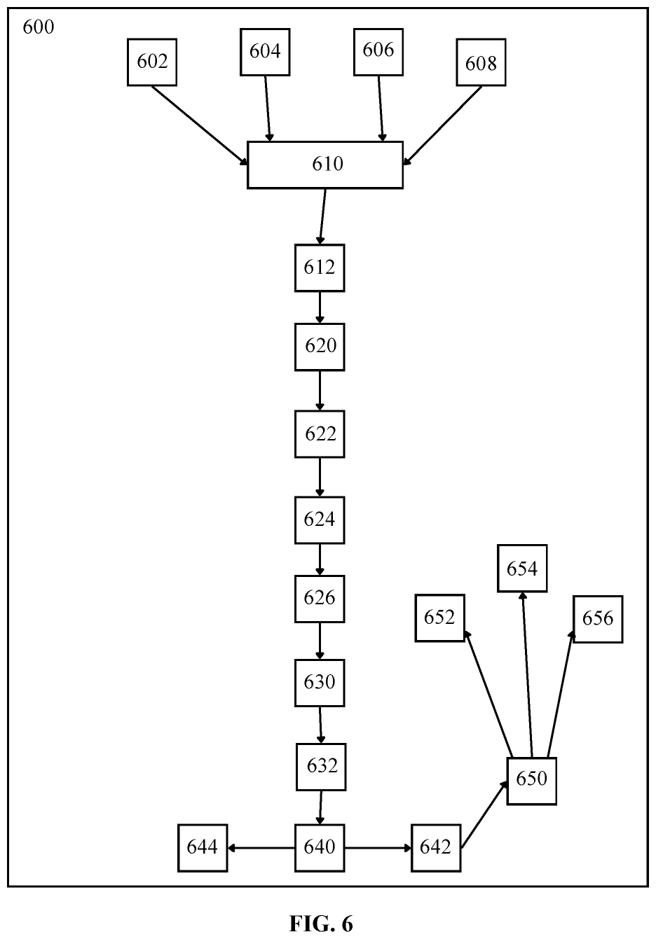

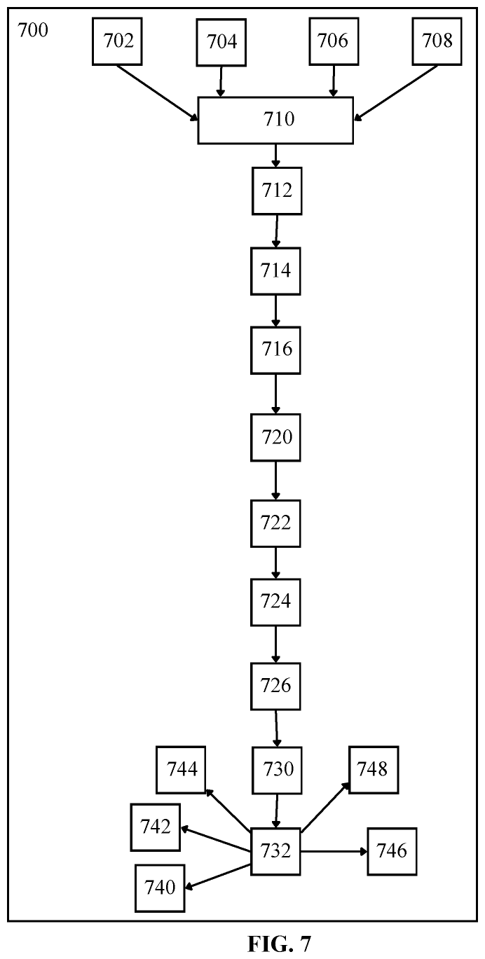

FIG. 6 illustrates an example recognition-enhanced predictive modeling workflow 600. In some embodiments, workflow 600 is used to generate one or more recognition-weighted activity-related predictions for one or more candidate compounds. In certain embodiments, workflow 600 applies a shared recognition-weighted framework to molecular descriptors, learned features, latent variables, grouped feature sets, or combinations thereof in order to improve robustness, reduce disproportionate influence from poorly calibrated inputs, and generate activity-related outputs suitable for ranking, filtering, prioritization, or downstream fusion.

In some embodiments, workflow 600 begins with receipt of one or more processed compound inputs 602, one or more processed descriptor inputs 604, one or more processed annotation inputs 606, and one or more modeling settings 608. In some embodiments, processed compound inputs 602 can include standardized compound structures, compound identifiers, conformer-linked records, or campaign-specific compound subsets. In some embodiments, processed descriptor inputs 604 can include physicochemical descriptors, topological descriptors, graph-derived descriptors, pharmacophore descriptors, learned embeddings, latent variables, fingerprints, quantum-chemical descriptors, or combinations thereof. In some embodiments, processed annotation inputs 606 can include measured activity values, binary activity labels, selectivity labels, potency values, assay class labels, confidence annotations, or combinations thereof. In some embodiments, modeling settings 608 can include model-selection parameters, feature-group definitions, normalization settings, weighting settings, confidence thresholds, or workflow-routing parameters.

In some embodiments, processed descriptor inputs 604 are routed to descriptor preparation step 610. In some embodiments, descriptor preparation step 610 includes missing-value handling, clipping, scaling, grouping, dimensionality reduction, latent projection, feature selection, redundancy reduction, variance filtering, confidence tagging, or combinations thereof. In some embodiments, descriptor preparation step 610 produces one or more prepared predictive inputs 612 suitable for subsequent recognition-enhanced modeling operations.

In some embodiments, workflow 600 can operate in a wrapper mode, a retrained mode, or a hybrid mode. In some embodiments, the wrapper mode applies recognition weighting to inputs, contributions, or outputs of an existing predictive model without requiring retraining of the underlying model. In some embodiments, the retrained mode uses recognition-weighted features, grouped recognition-weighted features, or recognition-derived features as inputs to train or fine-tune a predictive model. In some embodiments, the hybrid mode can apply recognition weighting both before and after one or more predictive modeling stages.

In some embodiments, prepared predictive inputs 612 are routed to recognition-metric derivation step 620. In some embodiments, step 620 derives one or more nonnegative dimensionless recognition metrics 622 from one or more descriptor values, grouped descriptor values, latent variables, embeddings, feature-family aggregates, or combinations thereof. In some embodiments, step 620 includes normalization relative to one or more selected scales, percentile-derived scales, descriptor-family scales, project-specific scales, target-specific scales, or other normalization bases.

In some embodiments, one or more inputs can be transformed before normalization through clipping, offsetting, absolute-value conversion, threshold conversion, bounded monotonic transformation, or combinations thereof.

In some embodiments, one or more nonnegative dimensionless recognition metrics 622 are processed at recognition coverage step 624 using the recognition coverage function F_cov(r)=r/(r+X_opt), where r is a nonnegative dimensionless recognition metric and X_opt is a constant. In certain embodiments, X_opt=phi/pi. In some embodiments, recognition coverage step 624 produces one or more bounded recognition-weight values 626 corresponding to one or more predictive inputs, feature groups, latent channels, or other modeling units.

In some embodiments, one or more prepared predictive inputs 612 and one or more bounded recognition-weight values 626 are combined at recognition-weighted feature generation step 630 to produce one or more recognition-weighted predictive inputs 632. In some embodiments, a recognition-weighted predictive input can correspond to a descriptor value multiplied by a bounded recognition-weight value. In some embodiments, a recognition-weighted predictive input can correspond to a grouped feature aggregate, a latent-variable contribution, a feature-family score, or another predictive quantity modulated using one or more bounded recognition-weight values.

In some embodiments, recognition-weighted predictive inputs 632 are routed to predictive model evaluation step 640. In some embodiments, predictive model evaluation step 640 can include application of a regression model, classification model, probabilistic model, neural network model, graph neural network model, ensemble model, support vector machine model, decision-tree-based model, random forest model, gradient boosting model, generalized linear model, or combinations thereof. In some embodiments, predictive model evaluation step 640 produces one or more activity-related predictions 642.

In some embodiments, the one or more activity-related predictions 642 can include predicted potency values, predicted binding values, predicted efficacy values, predicted selectivity values, predicted activity probabilities, predicted class assignments, uncertainty-aware predictions, confidence-weighted predictions, calibrated outputs, or combinations thereof. In some embodiments, activity-related predictions 642 can be associated with one target, multiple targets, one assay type, multiple assay types, or one or more target classes.

In some embodiments, workflow 600 can additionally or alternatively generate one or more contribution-level outputs 644. In some embodiments, contribution-level outputs 644 can identify one or more descriptors, feature groups, latent channels, or embeddings that contributed to a prediction. In some embodiments, contribution-level outputs 644 can be used to generate explanation data, feature-importance data, interpretability outputs, medicinal-chemistry guidance, or downstream filtering criteria.

In some embodiments, workflow 600 can implement a descriptor-level recognition-weighted prediction according to P_pred(C)=sum over n of alpha_n*D_n(C)*F_cov(D_n(C)), where alpha_n is a coefficient associated with descriptor n, D_n(C) is a descriptor-derived or transformed predictive input associated with candidate compound C, and F_cov(D_n(C)) is the bounded recognition-weight value derived from a corresponding nonnegative dimensionless recognition metric. In some embodiments, workflow 600 can alternatively use grouped descriptors, latent variables, embeddings, or non-linear combinations of predictive inputs rather than a simple linear form.

In some embodiments, one or more activity-related predictions 642 are processed at optional calibration and thresholding step 650. In some embodiments, step 650 includes score calibration, probability calibration, threshold comparison, uncertainty adjustment, confidence filtering, or combinations thereof. In some embodiments, calibration and thresholding step 650 produces one or more ranked predictive outputs 652, one or more classification outputs 654, one or more filtering decisions 656, or combinations thereof.

In some embodiments, workflow 600 can be used for potency prediction, binary activity prediction, selectivity prediction, off-target prediction, liability prediction, target-family activity prediction, phenotype-related prediction, or combinations thereof. In some embodiments, workflow 600 can be used in early virtual screening, hit expansion, lead optimization, scaffold prioritization, selectivity engineering, or portfolio-level triage.

In some embodiments, processed annotation inputs 606 are used to train, retrain, fine-tune, validate, calibrate, or benchmark one or more predictive models used in workflow 600. In some embodiments, processed annotation inputs 606 can additionally be used to define project-specific thresholds, guide feature selection, identify outlier regions, assign confidence levels, or support prospective validation of recognition-enhanced modeling outputs.

In some embodiments, workflow 600 can reduce disproportionate influence from poorly calibrated or weakly informative inputs by recognition-weighting one or more predictive inputs before final prediction generation. In some embodiments, this can attenuate the effect of extreme descriptor values, sparse feature patterns, outlier latent values, or project-misaligned signals that might otherwise dominate a conventional predictive model.

In some embodiments, workflow 600 is configurable with respect to descriptor choice, feature grouping, model type, wrapper or retrained operation, normalization strategy, calibration strategy, uncertainty handling, or output routing. In some embodiments, one or more operations of workflow 600 can be omitted, repeated, reordered, parallelized, subdivided, merged, or distributed across multiple computing resources. In some embodiments, workflow 600 can be executed as a stand-alone predictive subsystem, as part of an integrated multi-module screening platform, or in combination with molecular matching, structural optimization, developability analysis, composite ranking, or feedback-driven rescoring workflows.

In some embodiments, one or more outputs of workflow 600 are provided to recognition virtual screening ranking subsystem 160, user interface and reporting subsystem 170, feedback and model update workflow 1100, or one or more external analytics systems. In some embodiments, one or more outputs of workflow 600 can be logged, versioned, cached, exported, displayed, or associated with campaign-specific metadata for later review, medicinal-chemistry interpretation, model validation, or reanalysis.

By applying the shared recognition-weighted framework to predictive inputs, feature contributions, or modeled outputs, workflow 600 enables predictive modeling operations that are more robust to unstable, outlier, or weakly informative signals than conventional unweighted approaches. In some embodiments, this can improve prioritization quality, interpretability, and integration of predictive outputs within a broader computational drug discovery platform.

FIG. 7 illustrates an example recognition structural optimization workflow 700. In some embodiments, workflow 700 is used to generate one or more recognition-weighted structural optimization outputs for one or more candidate compounds. In certain embodiments, workflow 700 processes structural, conformational, energetic, geometric, interaction-based, or contact-derived information using the shared recognition framework in order to improve conformer selection, structural prioritization, fragment-linking analysis, linker evaluation, macrocycle assessment, preorganization analysis, or related structure-guided drug discovery operations.

In some embodiments, workflow 700 begins with receipt of one or more processed compound inputs 702, one or more processed target inputs 704, one or more conformer or pose inputs 706, and one or more structural optimization settings 708. In some embodiments, processed compound inputs 702 can include standardized structures, analog series members, fragment candidates, linker-bearing intermediates, macrocyclic candidates, or combinations thereof. In some embodiments, processed target inputs 704 can include prepared target structures, pocket definitions, residue annotations, hotspot definitions, or combinations thereof. In some embodiments, conformer or pose inputs 706 can include generated conformers, docked poses, aligned conformers, fragment placements, macrocycle geometries, or combinations thereof. In some embodiments, structural optimization settings 708 can include distance-scaling settings, interaction-channel settings, atom-group definitions, conformer-selection settings, or workflow routing preferences.

In some embodiments, one or more inputs 702, 704, and 706 are routed to interaction and geometry derivation step 710. In some embodiments, step 710 includes calculation or identification of one or more pairwise interaction values, pairwise distances, atom-group distances, contact values, pharmacophore distances, alignment values, interaction fingerprints, hotspot correspondence values, conformational strain indicators, torsional values, electrostatic terms, hydrogen-bond-related values, van der Waals-related values, desolvation-related values, pi-interaction-related values, metal-coordination-related values, or combinations thereof.

In some embodiments, interaction and geometry derivation step 710 produces one or more raw structural values 712. In some embodiments, raw structural values 712 can include signed energetic values, unsigned energetic magnitudes, geometric distances, correspondence values, alignment scores, contact-count values, structural penalties, or combinations thereof. In some embodiments, one or more raw structural values 712 are not directly suitable for use as nonnegative dimensionless recognition metrics and are therefore processed further.

In some embodiments, one or more raw structural values 712 are processed at structural-value transformation step 714 to generate one or more transformed structural values 716. In some embodiments, step 714 includes absolute-value conversion, negation, offsetting, clipping, reciprocal conversion, exponential conversion, monotonic distance-to-similarity conversion, penalty-to-benefit conversion, threshold conversion, bounded monotonic transformation, or combinations thereof. In some embodiments, transformed structural values 716 are selected such that larger values correspond to greater structural favorability, stronger target-relevant interaction quality, stronger preorganization, or other desired structural interpretation.

In some embodiments, one or more transformed structural values 716 are processed at recognition-metric derivation step 720 to generate one or more nonnegative dimensionless recognition metrics 722. In some embodiments, step 720 includes normalization relative to one or more selected scales, one or more reference distances, one or more project-defined normalization factors, one or more target-specific normalization factors, one or more interaction-class-specific normalization factors, or combinations thereof. In some embodiments, separate recognition metrics can be derived for separate interaction channels, atom-pair channels, residue-contact channels, pharmacophore channels, or conformational channels.

In some embodiments, one or more nonnegative dimensionless recognition metrics 722 are processed at recognition coverage step 724 using the recognition coverage function F_cov(r)=r/(r+X_opt), where r is a nonnegative dimensionless recognition metric and X_opt is a constant. In certain embodiments, X_opt=phi/pi. In some embodiments, recognition coverage step 724 produces one or more bounded recognition-weight values 726 corresponding to one or more structural features, interaction channels, or conformational channels.

In some embodiments, one or more transformed structural values 716 and one or more bounded recognition-weight values 726 are combined at structural optimization output step 730 to generate one or more recognition-weighted structural optimization outputs 732. In some embodiments, a recognition-weighted structural optimization output can be expressed as E_opt(C)=sum over i,j of E_ij*F_cov(d_ij), where E_ij is an interaction-related or structural value associated with components i and j of candidate compound C or of candidate compound C relative to a target-associated structural context, and d_ij is a corresponding nonnegative dimensionless recognition metric or normalized distance-derived value.

In some embodiments, E_ij can represent a pairwise energetic value, an interaction magnitude, a contact-derived value, a pharmacophore correspondence value, a structural compatibility value, or another structural term. In some embodiments, d_ij can represent a normalized distance, a transformed distance-derived correspondence value, a geometric proximity metric, a contact-density-derived value, or another nonnegative dimensionless recognition metric associated with the corresponding structural term.

In some embodiments, workflow 700 can generate one or more conformer rankings 740, one or more fragment-linking evaluations 742, one or more linker assessments 744, one or more macrocycle assessments 746, one or more preorganization outputs 748, or combinations thereof. In some embodiments, conformer rankings 740 can be used to select one or more conformers for docking, rescoring, pharmacophore comparison, or medicinal chemistry review. In some embodiments, fragment-linking evaluations 742 can be used to compare linker candidates or fragment arrangements. In some embodiments, macrocycle assessments 746 can be used to prioritize candidate macrocycles exhibiting favorable recognition-weighted structural characteristics.

In some embodiments, workflow 700 can additionally be used to assess hotspot occupancy, interaction-pattern quality, binding-mode stability, residue-contact favorability, scaffold geometry compatibility, or target-pocket compatibility. In some embodiments, workflow 700 can produce structural outputs for one target, multiple targets, one target family, or one or more off-targets in a selectivity workflow.

In some embodiments, workflow 700 can be applied before docking, after docking, during lead optimization, during fragment elaboration, during macrocycle design, during scaffold prioritization, or during iterative medicinal chemistry cycles. In some embodiments, one or more outputs of workflow 700 can be combined with docking-related outputs, predictive outputs, developability outputs, or feedback-derived outputs in downstream prioritization workflows.

In some embodiments, workflow 700 can process structural information at different granularities. For example, in some embodiments, workflow 700 can use atom-pair terms. In some embodiments, workflow 700 can use atom-to-group terms. In some embodiments, workflow 700 can use group-to-group terms, residue-contact terms, hotspot-region terms, or pharmacophore-feature terms. In some embodiments, different granularities can be used in parallel for the same candidate compound.

In some embodiments, workflow 700 is configurable with respect to structural input type, interaction channel selection, distance transformation method, normalization strategy, feature grouping, output type, or downstream routing. In some embodiments, one or more operations of workflow 700 can be omitted, repeated, reordered, parallelized, subdivided, merged, or distributed across multiple computing resources. In some embodiments, workflow 700 can be executed as a stand-alone structural module or in combination with molecular matching, predictive modeling, developability analysis, composite ranking, or feedback and update workflows.

In some embodiments, one or more outputs of workflow 700 are provided to recognition virtual screening ranking subsystem 160, user interface and reporting subsystem 170, feedback and model update workflow 1100, or one or more external structural analysis systems. In some embodiments, one or more outputs of workflow 700 can be stored, logged, versioned, exported, or associated with campaign-specific metadata for later review, medicinal chemistry interpretation, conformer reuse, or retrospective validation.

By applying the shared recognition-weighted framework to structural, geometric, and interaction-derived values, workflow 700 enables structural optimization operations that can attenuate weakly informative or poorly aligned structural contributions while preserving target-relevant structural favorability. In some embodiments, this can improve conformer selection, fragment-linking analysis, macrocycle prioritization, and integration of structural outputs with other computational drug discovery modules.

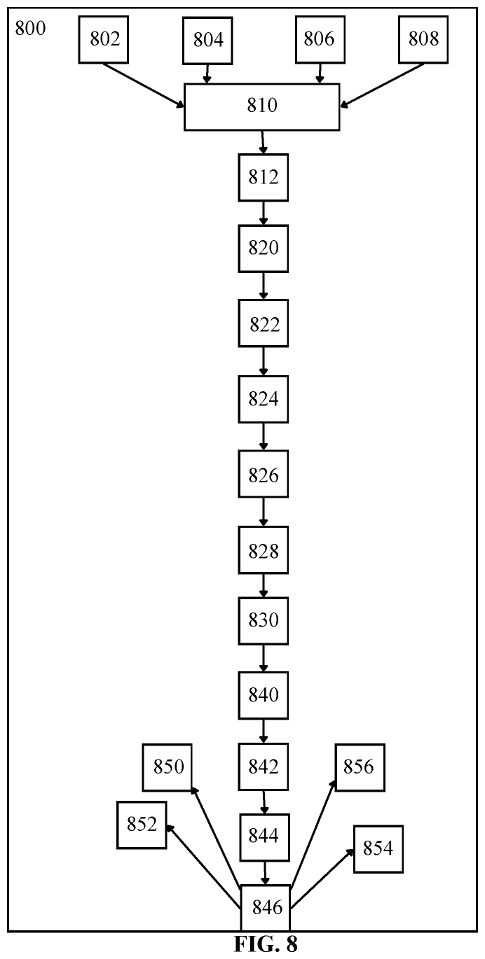

FIG. 8 illustrates an example recognition developability analysis workflow 800. In some embodiments, workflow 800 is used to generate one or more recognition-weighted developability outputs for one or more candidate compounds. In certain embodiments, workflow 800 processes physicochemical metrics, absorption-distribution-metabolism-excretion-toxicity-related metrics, safety-related metrics, formulation-related metrics, or combinations thereof using the shared recognition framework in order to support drug-likeness assessment, developability triage, filtering, prioritization, and downstream fusion with other scoring modules.

In some embodiments, workflow 800 begins with receipt of one or more processed compound inputs 802, one or more processed developability inputs 804, one or more optional annotation inputs 806, and one or more developability settings 808. In some embodiments, processed compound inputs 802 can include standardized compound structures, project-specific compound subsets, analog series members, or compound identifiers linked to one or more developability records. In some embodiments, processed developability inputs 804 can include physicochemical values, permeability-related values, solubility-related values, metabolic stability values, clearance-related values, protein-binding-related values, transporter-related values, toxicity-related values, formulation-related values, aggregation-related values, or combinations thereof. In some embodiments, optional annotation inputs 806 can include experimentally measured developability records, confidence indicators, assay-derived developability labels, literature-derived warnings, or combinations thereof. In some embodiments, developability settings 808 can include metric-selection settings, threshold settings, weighting settings, normalization settings, project-stage settings, or workflow-routing preferences.

In some embodiments, processed developability inputs 804 are routed to metric preparation step 810. In some embodiments, metric preparation step 810 includes unit harmonization, missing-value handling, clipping, outlier handling, confidence tagging, threshold assignment, target-window selection, directionality normalization, penalty conversion, or combinations thereof. In some embodiments, metric preparation step 810 produces one or more prepared developability metrics 812 suitable for subsequent recognition-weighted processing.

In some embodiments, prepared developability metrics 812 can include lipophilicity-related values, molecular size values, polarity values, hydrogen-bonding-related values, flexibility-related values, solubility values, permeability values, oral absorption values, plasma protein binding values, microsomal stability values, clearance values, cytochrome P450-related values, transporter-related values, blood-brain barrier values, hERG-related values, genotoxicity-related values, hepatotoxicity-related values, mitochondrial toxicity-related values, phototoxicity-related values, aggregation-related values, formulation-related values, or combinations thereof.

In some embodiments, one or more prepared developability metrics 812 are processed at developability transformation step 820 to generate one or more transformed developability values 822. In some embodiments, step 820 includes inversion of metrics for which lower raw values are more favorable, complement transformation, offsetting, clipping, target-window transformation, bounded monotonic transformation, penalty-to-benefit conversion, percentile-based conversion, or combinations thereof. In some embodiments, transformed developability values 822 are selected such that larger transformed values correspond to more favorable developability or project-stage suitability.

In some embodiments, one or more transformed developability values 822 are processed at recognition-metric derivation step 824 to generate one or more nonnegative dimensionless recognition metrics 826. In some embodiments, step 824 includes normalization relative to one or more selected scales, project-specific scales, target-class-specific scales, medicinal-chemistry-defined scales, percentile-derived scales, or combinations thereof. In some embodiments, separate recognition metrics can be generated for separate developability channels, safety channels, absorption channels, metabolism channels, or project-defined metric groups.

In some embodiments, one or more nonnegative dimensionless recognition metrics 826 are processed at recognition coverage step 828 using the recognition coverage function F_cov(r)=r/(r+X_opt), where r is a nonnegative dimensionless recognition metric and X_opt is a constant. In certain embodiments, X_opt=phi/pi. In some embodiments, recognition coverage step 828 produces one or more bounded recognition-weight values 830 corresponding to one or more developability metrics or metric groups.

In some embodiments, one or more transformed developability values 822 and one or more bounded recognition-weight values 830 are combined at developability aggregation step 840 to generate one or more intermediate developability values 842. In some embodiments, one or more intermediate developability values 842 are further combined at recognition developability output step 844 to generate one or more recognition-weighted developability outputs 846. In some embodiments, a recognition-weighted developability output can be expressed using M_drug_like(C)=sum over i of k_i*metric_i_norm(C), where k_i is a weighting coefficient associated with metric i and metric_i_norm(C) is a transformed or normalized developability-related value associated with candidate compound C, and S_ADMET(C)=F_cov(M_drug_like(C)).

In some embodiments, the weighting coefficients k_i can be fixed, project-specific, stage-specific, target-specific, medicinal-chemistry-defined, safety-defined, or learned from prior project outcomes. In some embodiments, workflow 800 can additionally or alternatively use grouped weights, hierarchical aggregation, gated aggregation, threshold-qualified aggregation, or combinations thereof.

In some embodiments, workflow 800 can generate one or more ranked developability outputs 850, one or more developability flags 852, one or more filtering decisions 854, one or more project-stage suitability outputs 856, or combinations thereof. In some embodiments, ranked developability outputs 850 can be used to prioritize compounds having favorable overall developability characteristics. In some embodiments, developability flags 852 can identify liabilities or threshold violations. In some embodiments, filtering decisions 854 can exclude compounds having insufficient recognition-weighted developability scores. In some embodiments, project-stage suitability outputs 856 can be used to distinguish compounds appropriate for exploratory screening from compounds appropriate for advanced optimization.

In some embodiments, workflow 800 can be used in early library triage, hit-to-lead progression, lead optimization, safety triage, formulation prioritization, selectivity campaigns, central nervous system programs, anti-infective programs, oncology programs, or other discovery settings. In some embodiments, workflow 800 can operate on experimentally measured developability data, predicted developability data, or combinations thereof.

In some embodiments, optional annotation inputs 806 can be used to calibrate, validate, benchmark, or explain one or more outputs of workflow 800. In some embodiments, optional annotation inputs 806 can identify confidence levels, assay-derived liabilities, literature-supported warnings, or prior medicinal-chemistry lessons relevant to one or more developability metrics or thresholds.

In some embodiments, workflow 800 can reduce fragmentation among multiple developability-related outputs by providing a bounded recognition-weighted developability output that is computationally consistent with the other recognition-weighted modules of platform 100. In some embodiments, this can simplify comparison between compounds across different discovery stages and can support integrated prioritization in combination with molecular matching, predictive modeling, and structural optimization outputs.

In some embodiments, workflow 800 is configurable with respect to metric selection, transformation choice, aggregation choice, weighting choice, thresholding strategy, annotation use, or downstream routing. In some embodiments, one or more operations of workflow 800 can be omitted, repeated, reordered, parallelized, subdivided, merged, or distributed across multiple computing resources. In some embodiments, workflow 800 can be executed as a stand-alone developability subsystem or in combination with one or more other modules of platform 100.

In some embodiments, one or more outputs of workflow 800 are provided to recognition virtual screening ranking subsystem 160, user interface and reporting subsystem 170, feedback and model update workflow 1100, or one or more external project management or medicinal-chemistry support systems. In some embodiments, one or more outputs of workflow 800 can be logged, versioned, cached, exported, displayed, or associated with campaign-specific metadata for later review, audit, or reanalysis.

By applying the shared recognition-weighted framework to developability-related metrics, workflow 800 enables bounded and computationally coherent developability analysis that can be integrated directly with the other recognition-weighted subsystems of platform 100. In some embodiments, this can improve prioritization quality, workflow consistency, and interpretability across drug discovery campaigns.

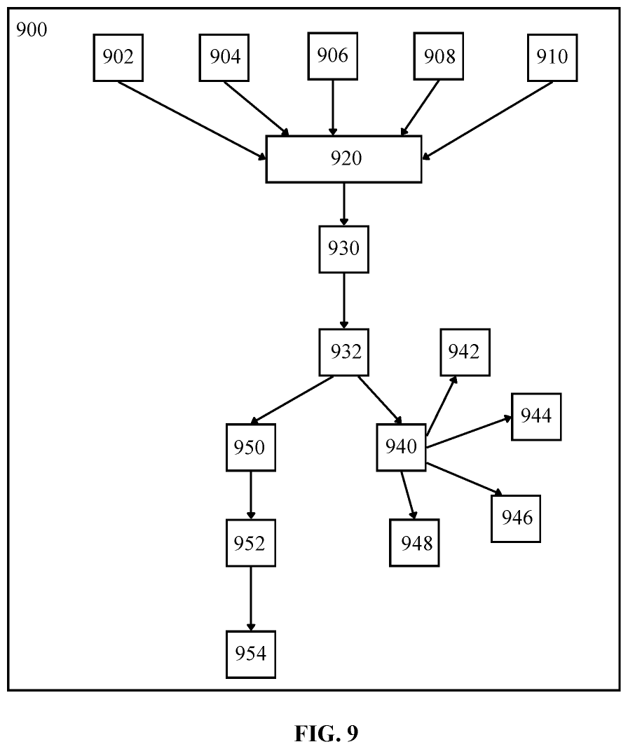

FIG. 9 illustrates an example composite virtual screening ranking workflow 900. In some embodiments, workflow 900 is used to combine outputs from two or more recognition-weighted subsystems in order to generate one or more composite prioritization outputs for one or more candidate compounds. In certain embodiments, workflow 900 integrates molecular matching outputs, predictive modeling outputs, structural optimization outputs, developability outputs, selectivity-related outputs, or combinations thereof to produce ranked, filtered, or otherwise ordered candidate sets for downstream decision-making.

In some embodiments, workflow 900 begins with receipt of one or more recognition-weighted molecular match outputs 902, one or more recognition-enhanced predictive outputs 904, one or more recognition-weighted structural optimization outputs 906, one or more recognition-weighted developability outputs 908, and one or more optional campaign or target-panel inputs 910. In some embodiments, one or more such inputs can be omitted depending on the configuration of a given discovery campaign. In some embodiments, workflow 900 combines outputs from two modules, three modules, four modules, or more than four modules.

In some embodiments, one or more received subsystem outputs are processed at scale harmonization step 920. In some embodiments, scale harmonization step 920 includes normalization, calibration, rank-based conversion, percentile conversion, clipping, bounded monotonic transformation, uncertainty adjustment, confidence weighting, or combinations thereof. In some embodiments, scale harmonization step 920 is used to place heterogeneous subsystem outputs into a common or compatible form suitable for downstream aggregation.

In some embodiments, scale harmonization step 920 can be configured on a per-module, per-target, per-project, per-campaign, or per-output-channel basis. For example, in some embodiments, molecular match outputs can be percentile-normalized while predictive outputs are probability-calibrated and developability outputs are threshold-adjusted. In some embodiments, structural optimization outputs can be converted into bounded or ranked forms before aggregation with other subsystem outputs.

In some embodiments, one or more harmonized outputs are processed at weighting and aggregation step 930 to generate one or more composite prioritization values 932. In some embodiments, weighting and aggregation step 930 uses fixed weights, project-specific weights, target-specific weights, campaign-stage weights, medicinal-chemistry-defined weights, learned weights, adaptive weights, or combinations thereof. In some embodiments, weighting and aggregation step 930 can use weighted sums, weighted averages, geometric means, harmonic means, rank fusion, gated aggregation, hierarchical aggregation, Pareto-based prioritization, voting-based aggregation, or combinations thereof.

In some embodiments, a composite prioritization value can be expressed as VS_score(C,T)=w1R_match(C,T)+w2P_pred(C)+w3*S_ADMET(C), where R_match(C,T) is a recognition-weighted molecular match output for candidate compound C relative to target T, P_pred(C) is a recognition-enhanced predictive output for candidate compound C, S_ADMET(C) is a recognition-weighted developability output for candidate compound C, and w1, w2, and w3 are weighting coefficients. In some embodiments, one or more structural optimization outputs or target-panel outputs can additionally be included in the composite prioritization value.

In some embodiments, one or more weighting coefficients can be selected to emphasize binding-related performance, predictive activity likelihood, selectivity, developability, structural compatibility, or other campaign objectives. In some embodiments, weighting coefficients can be dynamically adjusted based on campaign stage, target class, assay results, medicinal-chemistry feedback, or portfolio considerations.

In some embodiments, weighting coefficients are constrained such that each weighting coefficient is nonnegative and the coefficients sum to 1. In some embodiments, weighting and aggregation step 930 produces the composite prioritization value using one of: a weighted sum, a weighted average, a geometric mean, a minimum/maximum operator, or a rank-fusion operation. In some embodiments, prior to aggregation, each subsystem output is harmonized using a deterministic scale-harmonization rule including percentile conversion, z-score normalization, min-max scaling, or calibration to a reference distribution such that aggregated terms have comparable numeric meaning.

In some embodiments, composite prioritization values 932 are processed at ranking and selection step 940 to generate one or more ranked candidate lists 942, one or more filtered candidate sets 944, one or more deprioritized candidate sets 946, one or more diversity-aware selections 948, or combinations thereof. In some embodiments, ranked candidate lists 942 can be used to identify top candidate compounds for synthesis, acquisition, assay testing, or medicinal-chemistry review. In some embodiments, filtered candidate sets 944 can identify compounds meeting one or more minimum composite thresholds. In some embodiments, deprioritized candidate sets 946 can identify compounds failing to satisfy one or more desired composite objectives.

In some embodiments, diversity-aware selections 948 can be generated by applying one or more scaffold-diversity constraints, chemotype-diversity constraints, cluster-based selection procedures, novelty constraints, or budget-based selection rules after or during composite ranking. In some embodiments, such diversity-aware selections can be used to reduce redundancy among top-ranked compounds while preserving favorable composite prioritization values.

In some embodiments, workflow 900 can additionally generate one or more explanation outputs 950, one or more score-breakdown outputs 952, one or more threshold-qualification outputs 954, or combinations thereof. In some embodiments, explanation outputs 950 can identify module-specific contributions to a final ranking. In some embodiments, score-breakdown outputs 952 can provide per-compound or per-target summaries showing how subsystem outputs contributed to composite prioritization. In some embodiments, threshold-qualification outputs 954 can indicate whether a compound satisfied one or more project-defined selection criteria.

In some embodiments, a scale used to normalize a transformed value into a nonnegative dimensionless recognition metric is selected according to a deterministic rule. For example, a selected scale can be (i) a target-specific percentile (e.g., a 90th or 95th percentile) of the transformed values observed over a screening library, (ii) a reference-derived scale computed from one or more reference ligands or reference-associated compounds, or (iii) a fixed project configuration value stored in a model or parameter store. In some embodiments, a compound is selected for a follow-on action when a composite prioritization value exceeds a configurable threshold or falls within a top-K ranked set, where the threshold and K are stored as workflow settings and logged with the screening output.

In some embodiments, workflow 900 can be used in library triage, hit identification, hit expansion, lead optimization, selectivity optimization, off-target risk management, target-panel assessment, scaffold prioritization, or portfolio-level screening programs. In some embodiments, workflow 900 can be applied to a full screening library, a focused medicinal-chemistry set, a fragment set, a macrocycle set, an enumerated virtual library, or combinations thereof.

In some embodiments, one or more optional campaign or target-panel inputs 910 can include target-priority weights, off-target penalty settings, budget constraints, stage-specific screening rules, diversity constraints, selectivity objectives, or medicinal-chemistry preferences. In some embodiments, such inputs can be incorporated at scale harmonization step 920, weighting and aggregation step 930, ranking and selection step 940, or combinations thereof.

In some embodiments, workflow 900 can be configured to produce one ranking for a primary target, multiple rankings for multiple targets, a selectivity-aware ranking, an off-target-penalized ranking, a developability-prioritized ranking, or other specialized prioritization outputs. In some embodiments, workflow 900 can additionally produce separate worklists for assay testing, structural review, medicinal-chemistry review, safety review, or iterative computational follow-up.

In some embodiments, workflow 900 is configurable with respect to module inclusion, scale harmonization method, weighting method, aggregation method, thresholding method, diversity-selection method, explanation method, or downstream routing. In some embodiments, one or more operations of workflow 900 can be omitted, repeated, reordered, parallelized, subdivided, merged, or distributed across multiple computing resources. In some embodiments, workflow 900 can be executed as a stand-alone ranking subsystem or as part of an integrated screening platform.

In some embodiments, one or more outputs of workflow 900 are provided to user interface and reporting subsystem 170, feedback and model update workflow 1100, one or more external laboratory information systems, one or more medicinal-chemistry planning systems, or one or more portfolio-management systems. In some embodiments, one or more outputs of workflow 900 can be logged, versioned, cached, exported, displayed, or associated with campaign-specific metadata for later validation, auditing, campaign comparison, or retrospective benchmarking.

By combining outputs from multiple recognition-weighted subsystems into one or more composite prioritization values, workflow 900 enables integrated candidate ranking that can reflect binding-related fit, predictive activity, structural favorability, and developability within a common platform. In some embodiments, this can improve prioritization quality, reduce reliance on ad hoc manual weighting, and support more consistent drug discovery decision-making across campaigns.

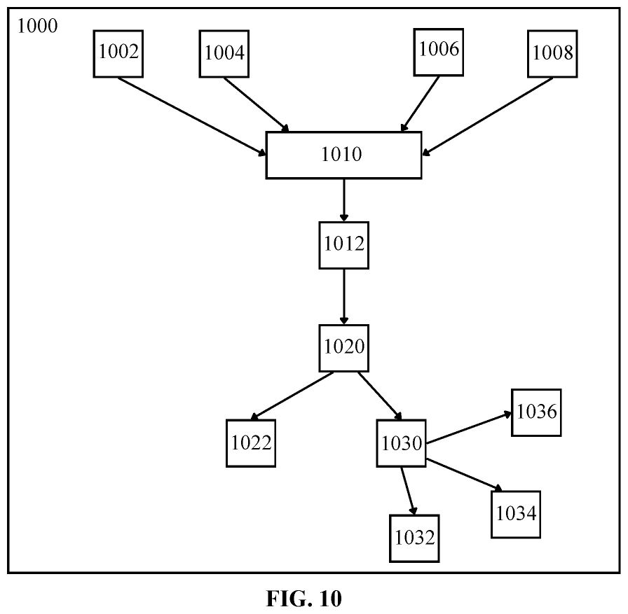

FIG. 10 illustrates an example multi-target and selectivity workflow 1000. In some embodiments, workflow 1000 is used to evaluate one or more candidate compounds relative to multiple targets in order to generate one or more target-preference, selectivity, polypharmacology, or off-target-risk outputs. In certain embodiments, workflow 1000 applies one or more recognition-weighted scoring operations across a target panel so that candidate compounds can be prioritized not only for favorable interaction with a primary target but also for favorable selectivity relative to one or more secondary targets, anti-targets, or off-targets.

In some embodiments, workflow 1000 begins with receipt of one or more candidate compound inputs 1002, one or more primary target inputs 1004, one or more secondary target or off-target inputs 1006, and one or more target-associated reference inputs 1008. In some embodiments, candidate compound inputs 1002 can include one or more compounds, analog series members, fragments, macrocycles, covalent candidates, or other candidate structures to be compared across multiple targets. In some embodiments, primary target inputs 1004 can correspond to one or more intended therapeutic targets, while secondary target or off-target inputs 1006 can correspond to one or more related targets, safety-relevant targets, counter-screen targets, anti-targets, or other targets relevant to selectivity assessment. In some embodiments, target-associated reference inputs 1008 can include one or more known ligands, co-crystallized ligands, selective binders, pharmacophore templates, interaction fingerprints, shape models, electrostatic models, or other reference-associated representations corresponding to one or more targets in the target panel.

In some embodiments, one or more target-specific scoring operations 1010 are performed for each target in the panel. In some embodiments, a target-specific scoring operation 1010 includes one or more molecular matching operations, predictive modeling operations, structural optimization operations, developability-related operations, or combinations thereof, performed with respect to a corresponding target context. In some embodiments, the one or more target-specific scoring operations 1010 produce one or more target-specific recognition-weighted outputs 1012 for each candidate compound with respect to each evaluated target.

In some embodiments, one or more target-specific recognition-weighted outputs 1012 include one or more recognition-weighted molecular match outputs, one or more target-specific predictive outputs, one or more target-specific structural outputs, or combinations thereof. In some embodiments, the target-specific outputs can be generated using target-specific reference ligands, target-specific pharmacophore models, target-specific docking grids, target-specific descriptor models, target-specific thresholds, target-specific weighting parameters, or combinations thereof.

In some embodiments, the one or more target-specific recognition-weighted outputs 1012 are processed at selectivity derivation step 1020. In some embodiments, selectivity derivation step 1020 includes comparison of one or more primary-target outputs with one or more secondary-target or off-target outputs. In some embodiments, step 1020 includes generation of one or more ratios, differences, margins, weighted panel scores, penalized scores, threshold-qualified scores, or combinations thereof.

In some embodiments, a selectivity-related value can be expressed as SelectivityScore(C)=R_match(C,Target_A)/R_match(C,Target_B), where R_match(C,Target_A) is a recognition-weighted molecular match output for candidate compound C relative to a first target and R_match(C,Target_B) is a recognition-weighted molecular match output for candidate compound C relative to a second target. In some embodiments, selectivity can additionally or alternatively be expressed using differences, weighted differences, log ratios, normalized panel contrasts, composite target-versus-off-target scores, or other comparison functions.

In some embodiments, workflow 1000 can additionally generate one or more panel scores 1022. In some embodiments, a panel score can be expressed as S_panel(C)=sum over t of w_t*R_match(C,t), where w_t is a target-specific weight and R_match(C,t) is a recognition-weighted output for candidate compound C relative to target t. In some embodiments, the panel score can reward desired target interactions, penalize undesired target interactions, or combine both effects into a single target-panel-oriented prioritization value.

In some embodiments, target-specific weights can be fixed, project-specific, safety-driven, selectivity-driven, indication-specific, or learned from prior campaign outcomes. In some embodiments, one or more target-specific weights can emphasize a primary therapeutic target while penalizing one or more off-targets associated with safety liabilities, poor tolerability, or mechanistic undesirability.

In some embodiments, one or more selectivity-related values or panel scores are processed at target-panel prioritization step 1030 to generate one or more selectivity-ranked candidate lists 1032, one or more off-target-risk flags 1034, one or more polypharmacology profiles 1036, or combinations thereof. In some embodiments, selectivity-ranked candidate lists 1032 can identify compounds exhibiting favorable recognition-weighted preference for one or more primary targets relative to one or more secondary targets. In some embodiments, off-target-risk flags 1034 can identify compounds exhibiting undesired recognition-weighted affinity patterns across one or more counter-screen or anti-target panels. In some embodiments, polypharmacology profiles 1036 can summarize recognition-weighted target engagement patterns across a multi-target panel.

In some embodiments, workflow 1000 can be used for GPCR subtype selectivity, kinase family selectivity, ion-channel selectivity, protease family selectivity, transporter selectivity, nuclear receptor selectivity, protein-protein interaction selectivity, RNA target selectivity, anti-infective target panel screening, oncology target panel screening, central nervous system safety screening, cardiovascular liability screening, or combinations thereof.

In some embodiments, workflow 1000 can be configured for one-to-one target comparisons, one-to-many target comparisons, many-to-many target comparisons, or hierarchical target-family analyses. In some embodiments, workflow 1000 can compare one primary target against one off-target, one primary target against a panel of off-targets, multiple desired targets against multiple undesired targets, or one or more target families against one or more other target families.

In some embodiments, workflow 1000 can incorporate one or more target-specific thresholds, minimum selectivity margins, maximum off-target penalties, panel diversity constraints, or stage-specific project rules. In some embodiments, such criteria can be applied before, during, or after selectivity derivation step 1020 or target-panel prioritization step 1030.

In some embodiments, one or more outputs of workflow 1000 can be combined with developability outputs, predictive outputs, structural optimization outputs, or campaign-level prioritization outputs in composite ranking workflow 900. In some embodiments, workflow 1000 can operate as a specialized selectivity module within platform 100 or as a target-panel extension of one or more other disclosed subsystems.

In some embodiments, workflow 1000 is configurable with respect to target-panel composition, target-specific reference selection, scoring-module inclusion, selectivity formula choice, penalty structure, target-weighting strategy, thresholding strategy, or downstream routing. In some embodiments, one or more operations of workflow 1000 can be omitted, repeated, reordered, parallelized, subdivided, merged, or distributed across multiple computing resources.

In some embodiments, one or more outputs of workflow 1000 are provided to recognition virtual screening ranking subsystem 160, user interface and reporting subsystem 170, feedback and model update workflow 1100, one or more medicinal-chemistry planning systems, one or more assay-planning systems, or one or more portfolio-management systems. In some embodiments, one or more outputs of workflow 1000 can be logged, versioned, cached, exported, displayed, or associated with target-panel metadata for later review, selectivity benchmarking, or iterative project refinement.

By applying recognition-weighted scoring across multiple targets and deriving one or more selectivity-related or panel-level outputs, workflow 1000 enables target prioritization decisions that can account for both desired target engagement and undesired off-target interaction risk. In some embodiments, this can improve candidate selection for programs in which target specificity, subtype discrimination, safety profiling, or polypharmacology balancing is important.

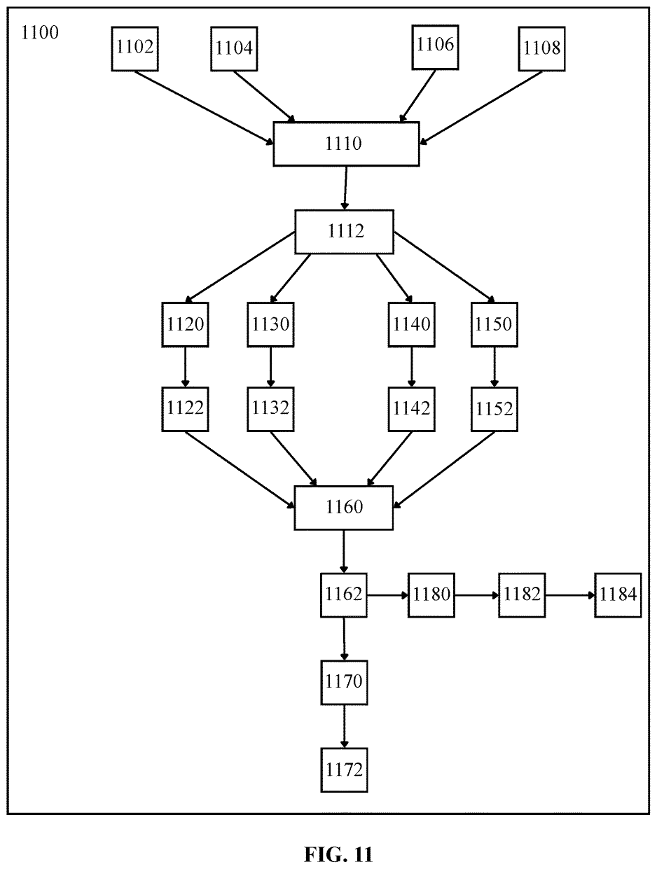

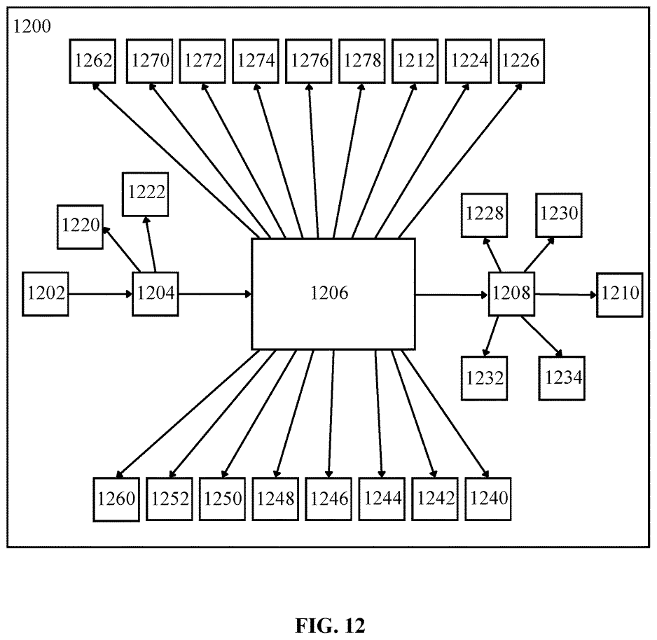

FIG. 11 illustrates an example feedback and model update workflow 1100. In some embodiments, workflow 1100 is used to incorporate assay results, medicinal chemistry review, screening outcomes, target-panel observations, or other campaign-related information into one or more updated parameters, references, thresholds, weights, models, or workflow settings of platform 100. In certain embodiments, workflow 1100 enables iterative refinement of one or more recognition-weighted subsystems while preserving the shared recognition framework described herein.

In some embodiments, workflow 1100 begins with receipt of one or more screening outputs 1102, one or more assay-derived results 1104, one or more medicinal chemistry review inputs 1106, and one or more campaign analysis inputs 1108. In some embodiments, screening outputs 1102 can include ranked candidate lists, composite prioritization outputs, selectivity outputs, module-specific scores, explanation outputs, or combinations thereof produced by one or more prior runs of platform 100. In some embodiments, assay-derived results 1104 can include potency results, binding results, selectivity results, toxicity results, permeability results, metabolism results, formulation results, or other experimental outcomes associated with one or more candidate compounds. In some embodiments, medicinal chemistry review inputs 1106 can include expert annotations, scaffold preferences, liability assessments, synthesis feasibility observations, analog-series insights, or other project-specific review information. In some embodiments, campaign analysis inputs 1108 can include hit-rate summaries, false-positive analyses, structure-activity relationship observations, project-stage transitions, target-priority changes, benchmark comparisons, or combinations thereof.

In some embodiments, one or more received inputs are processed at feedback consolidation step 1110. In some embodiments, feedback consolidation step 1110 includes matching new results to prior candidate records, aligning assay outcomes with computational predictions, associating medicinal chemistry annotations with scored compounds, computing project-level statistics, identifying mis-ranked compounds, identifying false positives or false negatives, identifying selectivity failures, identifying developability failures, or combinations thereof. In some embodiments, feedback consolidation step 1110 produces one or more consolidated feedback records 1112.

In some embodiments, one or more consolidated feedback records 1112 are processed at parameter update step 1120. In some embodiments, parameter update step 1120 includes updating one or more weighting coefficients, updating one or more thresholds, updating one or more normalization factors, updating one or more scale harmonization settings, updating one or more module-routing settings, updating one or more confidence filters, or combinations thereof. In some embodiments, parameter update step 1120 produces one or more updated parameter sets 1122.

In some embodiments, one or more consolidated feedback records 1112 are additionally or alternatively processed at reference update step 1130. In some embodiments, reference update step 1130 includes adding one or more new reference ligands, removing one or more poor-performing references, reweighting one or more references, updating one or more pharmacophore models, updating one or more interaction fingerprints, updating one or more target-specific reference ensembles, or combinations thereof. In some embodiments, reference update step 1130 produces one or more updated reference sets 1132 for use in subsequent molecular matching or target-panel workflows.

In some embodiments, one or more consolidated feedback records 1112 are additionally or alternatively processed at model update step 1140. In some embodiments, model update step 1140 includes retraining one or more predictive models, fine-tuning one or more predictive models, recalibrating one or more predictive outputs, updating one or more uncertainty models, updating one or more feature-selection settings, updating one or more descriptor-group definitions, or combinations thereof. In some embodiments, model update step 1140 produces one or more updated model assets 1142.

In some embodiments, one or more consolidated feedback records 1112 are additionally or alternatively processed at workflow policy update step 1150. In some embodiments, workflow policy update step 1150 includes updating one or more stage-specific routing rules, updating one or more diversity-selection policies, updating one or more target-panel policies, updating one or more assay-selection criteria, updating one or more project-stage suitability rules, or combinations thereof. In some embodiments, workflow policy update step 1150 produces one or more updated workflow settings 1152.

In some embodiments, one or more updated parameter sets 1122, updated reference sets 1132, updated model assets 1142, and updated workflow settings 1152 are processed at validation and benchmarking step 1160. In some embodiments, validation and benchmarking step 1160 includes retrospective validation, holdout evaluation, scaffold-split evaluation, external validation, hit-rate analysis, enrichment analysis, ranking-quality analysis, calibration analysis, selectivity analysis, or combinations thereof. In some embodiments, validation and benchmarking step 1160 produces one or more update validation outputs 1162 indicating whether a proposed update should be accepted, modified, rejected, or conditionally deployed.

In some embodiments, one or more accepted updates are deployed at deployment step 1170. In some embodiments, deployment step 1170 includes publishing one or more updated parameters, activating one or more updated reference sets, switching one or more predictive models, updating one or more user-facing settings, updating one or more target-panel configurations, or combinations thereof. In some embodiments, deployment step 1170 produces one or more updated platform configurations 1172 for subsequent screening campaigns or iterative rescoring operations.

In some embodiments, workflow 1100 can additionally generate one or more audit outputs 1180, one or more explanation updates 1182, one or more benchmark reports 1184, or combinations thereof. In some embodiments, audit outputs 1180 can identify which parameters, references, models, or policies changed and why. In some embodiments, explanation updates 1182 can reflect changed feature importance, changed target-reference relationships, or changed threshold logic. In some embodiments, benchmark reports 1184 can summarize differences in hit rate, selectivity quality, ranking quality, developability filtering, or other campaign-relevant outcomes before and after one or more updates.

In some embodiments, workflow 1100 can operate continuously, periodically, after each assay batch, after each screening round, after each medicinal chemistry review cycle, after one or more project-stage transitions, or on another scheduled or event-triggered basis. In some embodiments, some updates can be performed automatically while other updates require user review, administrative approval, scientific review, or combinations thereof.

In some embodiments, workflow 1100 is configurable with respect to which feedback sources are used, which update types are permitted, which validation criteria are required, which deployment rules are applied, or how update records are stored and exposed to users. In some embodiments, one or more operations of workflow 1100 can be omitted, repeated, reordered, parallelized, subdivided, merged, or distributed across multiple computing resources.

In some embodiments, workflow 1100 can be used to improve molecular matching references, improve predictive modeling calibration, improve developability thresholds, improve selectivity prioritization, improve campaign-specific weighting, or combinations thereof. In some embodiments, workflow 1100 can also be used to adapt platform behavior across different target classes, different therapeutic areas, different project stages, or different organizational workflows.

By incorporating assay-derived outcomes, medicinal chemistry feedback, and campaign-level observations into updated parameters, references, models, and workflow policies, workflow 1100 enables iterative refinement of platform 100 while maintaining the shared recognition-weighted computational basis described herein. In some embodiments, this can improve future prioritization quality, reduce repeated false positives, and better align computational outputs with project-specific discovery objectives.

FIG. 12 illustrates an example computing environment 1200 for implementing platform 100 and the associated workflows described herein. In some embodiments, computing environment 1200 includes one or more client devices 1202, one or more application servers 1204, one or more model execution or workflow orchestration servers 1206, one or more data stores 1208, one or more external tool resources 1210, and one or more network connections 1212 enabling communication among such components.

In some embodiments, client devices 1202 can include desktop computers, laptop computers, workstation systems, tablet devices, mobile devices, thin clients, remote terminals, or other user-accessible computing devices through which a user can configure workflows, submit screening jobs, review ranked outputs, inspect score breakdowns, or otherwise interact with platform 100. In some embodiments, client devices 1202 can present one or more graphical user interfaces, dashboards, report-generation interfaces, application programming interfaces, or command-line interfaces.

In some embodiments, application servers 1204 host one or more user-facing services, workflow management services, job submission services, authentication services, authorization services, reporting services, or combinations thereof. In some embodiments, application servers 1204 receive screening requests, target-panel requests, prioritization requests, rescoring requests, or update requests from one or more client devices 1202 and route corresponding tasks to one or more downstream computational components.

In some embodiments, model execution or workflow orchestration servers 1206 execute one or more computational modules of platform 100, including data ingestion and preprocessing subsystem 110, recognition molecular matching subsystem 120, recognition-enhanced predictive modeling subsystem 130, recognition structural optimization subsystem 140, recognition developability analysis subsystem 150, recognition virtual screening ranking subsystem 160, multi-target and selectivity workflow 1000, feedback and model update workflow 1100, or combinations thereof. In some embodiments, servers 1206 schedule tasks, manage queues, allocate computational resources, coordinate module execution order, monitor job progress, or perform distributed workflow control.

In some embodiments, data stores 1208 include one or more compound structure databases 1220, one or more target structure databases 1222, one or more reference ligand and reference-model databases 1224, one or more descriptor and feature databases 1226, one or more developability and assay databases 1228, one or more model and parameter stores 1230, one or more workflow result stores 1232, and one or more audit and logging stores 1234. In some embodiments, one or more of data stores 1208 can be implemented using relational databases, document stores, object stores, graph stores, flat files, versioned repositories, or combinations thereof.

In some embodiments, external tool resources 1210 include one or more docking engines 1240, one or more cheminformatics engines 1242, one or more descriptor-generation engines 1244, one or more molecular mechanics or simulation engines 1246, one or more machine learning frameworks 1248, one or more external database interfaces 1250, one or more assay-data interfaces 1252, or combinations thereof. In some embodiments, external tool resources 1210 can be local to an organizational network, remotely hosted, cloud-hosted, vendor-hosted, or distributed across multiple environments.

In some embodiments, network connections 1212 include one or more local area networks, wide area networks, secure organizational networks, virtual private networks, wireless networks, cloud networks, internet-based connections, or combinations thereof. In some embodiments, one or more components of computing environment 1200 communicate using encrypted channels, authenticated application programming interfaces, message queues, file-based transfer mechanisms, streaming interfaces, or combinations thereof.

In some embodiments, one or more processors 1260 execute instructions stored in one or more memory devices 1262 to perform the operations described herein. In some embodiments, processors 1260 can include one or more central processing units, graphics processing units, tensor-processing units, vector processors, field-programmable gate arrays, application-specific accelerators, or combinations thereof. In some embodiments, memory devices 1262 can include volatile memory, non-volatile memory, local memory, distributed memory, cache memory, persistent storage, or combinations thereof.

In some embodiments, computing environment 1200 can support local execution, remote execution, distributed execution, hybrid execution, cloud-based execution, on-premises execution, or combinations thereof. In some embodiments, some modules of platform 100 can execute on local workstation resources while other modules execute on remote servers, cluster resources, cloud resources, or accelerator-backed infrastructure. In some embodiments, high-throughput docking operations, vectorized recognition processing, predictive-model inference, or batch ranking operations can be distributed across multiple compute nodes.

In some embodiments, platform 100 is implemented as software stored in a non-transitory computer-readable medium and executed by one or more processors. In some embodiments, the non-transitory computer-readable medium stores instructions that, when executed by one or more processors, cause the one or more processors to receive one or more candidate compound inputs, one or more target-related inputs, one or more reference-related inputs, one or more descriptor-related inputs, one or more developability-related inputs, or combinations thereof; derive one or more nonnegative dimensionless recognition metrics; apply the recognition coverage function F_cov(r)=r/(r+X_opt), where X_opt is a constant and, in certain embodiments, X_opt=phi/pi; generate one or more recognition-weighted outputs; and produce one or more ranked, filtered, selected, deprioritized, or optimized candidate compound outputs.

In some embodiments, computing environment 1200 further includes one or more queue managers 1270, one or more job schedulers 1272, one or more containerized execution environments 1274, one or more workflow policy managers 1276, and one or more monitoring components 1278. In some embodiments, queue managers 1270 and job schedulers 1272 coordinate execution of large screening tasks, target-panel analyses, or rescoring campaigns across multiple compute resources. In some embodiments, containerized execution environments 1274 package one or more modules or dependencies for reproducible deployment. In some embodiments, workflow policy managers 1276 control access to target-specific settings, project-stage rules, model versions, or threshold configurations. In some embodiments, monitoring components 1278 track task status, performance, failure conditions, throughput, or resource utilization.

In some embodiments, computing environment 1200 supports one or more user-specific, team-specific, project-specific, or enterprise-specific configurations. In some embodiments, access controls can limit which compound libraries, target datasets, reference sets, predictive models, or scoring configurations are available to a given user or organizational group. In some embodiments, audit and logging stores 1234 can retain records sufficient to reconstruct which inputs, models, references, thresholds, and outputs were used in a given screening run or update cycle.

In some embodiments, computing environment 1200 supports export and interoperability functions. In some embodiments, ranked outputs, score breakdowns, reference associations, target-panel reports, developability reports, selectivity reports, benchmark summaries, or update records can be exported in one or more tabular formats, structure-data formats, report formats, application programming interface responses, or combinations thereof. In some embodiments, such exports can be provided to assay-planning systems, medicinal-chemistry systems, electronic laboratory notebooks, laboratory information management systems, portfolio-management systems, or other downstream user environments.

In some embodiments, one or more components of computing environment 1200 can be replicated, load-balanced, mirrored, sharded, cached, versioned, or geographically distributed. In some embodiments, the architecture can support scale-out library screening, repeated campaign rescoring, multi-target panel evaluation, and iterative model or reference updates while preserving traceability of inputs and outputs. In some embodiments, failover mechanisms, backup mechanisms, and version-control mechanisms can be used to preserve continuity and auditability.

In some embodiments, the computing environment shown in FIG. 12 is illustrative and not limiting. One or more components can be omitted, combined, subdivided, replaced, or implemented differently. For example, in some embodiments, a single workstation can implement one or more portions of platform 100. In some embodiments, a cloud-based deployment can implement most or all platform functions across multiple remote services. In some embodiments, one or more external tool resources 1210 can be integrated directly into application servers 1204 or workflow orchestration servers 1206 rather than being maintained as separate resources.

By providing an implementation architecture that supports user interaction, workflow execution, storage, external-tool interoperability, distributed compute, and non-transitory computer-readable medium embodiments, computing environment 1200 enables practical deployment of the recognition-optimized drug discovery platform across a wide range of organizational and technical settings.

In some embodiments, the disclosed platform can be applied to hit identification campaigns, hit expansion campaigns, lead optimization campaigns, selectivity engineering programs, off-target liability reduction programs, fragment-based discovery programs, macrocycle design programs, protein-protein interaction modulation programs, nucleic-acid targeting programs, anti-infective discovery programs, oncology discovery programs, central nervous system programs, rare disease programs, personalized-medicine workflows, or combinations thereof.

In some embodiments, the disclosed platform can be used with one or more public data sources, private organizational data sources, experimentally measured datasets, predicted datasets, literature-derived datasets, or combinations thereof. In some embodiments, one or more target-specific workflows can be configured using project-specific references, project-specific thresholds, project-specific weights, project-specific developability criteria, or project-specific stage gates.

In some embodiments, one or more of the disclosed subsystems can operate independently. In some embodiments, recognition molecular matching subsystem 120 can be used without recognition-enhanced predictive modeling subsystem 130. In some embodiments, recognition developability analysis subsystem 150 can be used as a stand-alone filtering system. In some embodiments, recognition virtual screening ranking subsystem 160 can combine only two or three modules rather than all modules. In some embodiments, multi-target workflow 1000 can be used without feedback and model update workflow 1100. In some embodiments, the shared recognition processing framework 500 can be used to support additional computational drug discovery modules not expressly illustrated in the figures.

In some embodiments, the disclosed methods and systems provide a unified recognition-weighted computational framework that can transform heterogeneous drug discovery inputs into bounded, interpretable, and campaign-configurable outputs. In some embodiments, use of the shared recognition coverage function across multiple computational modules can improve consistency between subsystem outputs, facilitate integrated prioritization, reduce reliance on ad hoc manual weighting, and support more robust identification and ranking of candidate compounds.

Although specific embodiments have been described herein for purposes of illustration, the disclosure is not limited to the described embodiments. A person of ordinary skill in the art, having the benefit of this disclosure, will appreciate that various modifications, substitutions, rearrangements, additions, and omissions can be made without departing from the spirit and scope of the invention as defined by the claims.

The following examples are provided to illustrate representative uses of the disclosed recognition-optimized drug discovery platform. The examples are intended to be illustrative and not limiting. A person of ordinary skill in the art, having the benefit of this disclosure, will appreciate that the disclosed workflows can be adapted to different target classes, compound sets, scoring configurations, campaign settings, and project objectives.

In the examples below, one or more candidate compounds are processed using one or more of the disclosed recognition-weighted subsystems, including recognition molecular matching, recognition-enhanced predictive modeling, recognition structural optimization, recognition developability analysis, composite virtual screening ranking, and multi-target selectivity analysis. In some embodiments, one or more raw or intermediate values are transformed into one or more nonnegative dimensionless recognition metrics and then processed using the recognition coverage function F_cov(r)=r/(r+X_opt), where X_opt is a constant and, in certain embodiments, X_opt=phi/pi.

In some embodiments, the examples use experimentally measured data, predicted data, public data, private project data, literature-derived data, simulated data, prospective campaign data, retrospective campaign data, or combinations thereof. In some embodiments, the examples are prophetic examples illustrating representative use of the disclosed platform in practical discovery settings.

In one embodiment, the disclosed platform is used in a kinase inhibitor screening campaign directed to a protein kinase target associated with an oncology indication. In some embodiments, a compound library including commercially available kinase-focused compounds, internal analog series, and enumerated analog candidates is received and standardized as described herein. A prepared target structure is generated using an experimentally determined kinase structure including a defined ATP-binding pocket, and one or more known active kinase inhibitors and one or more co-crystallized ligands are selected as reference inputs for recognition molecular matching. By way of a non-limiting example, a transformed docking value DockScore(C,T) may be set to 12.0 for a candidate compound C relative to target T. A similarity-related value may be normalized to produce a recognition metric r=0.80. Using X_opt=phi/pi, the recognition coverage function yields F_cov(r)=r/(r+X_opt). The recognition-weighted molecular match score can then be computed as R_match(C,T)=DockScore(C,T)*F_cov(r). In some embodiments, compounds are advanced when R_match(C,T) exceeds a stored threshold or when R_match(C, T) is within a top-K set for the target.

In some embodiments, candidate compounds are docked into the prepared kinase binding site to generate one or more docking-related values. In some embodiments, one or more structural similarity values are computed between each docked candidate compound and one or more selected reference ligands using one or more fingerprint, pharmacophore, shape, electrostatic, or interaction-pattern similarity measures. In some embodiments, the docking-related values are transformed so that higher transformed values correspond to more favorable docking-related outcomes, and one or more normalized similarity-derived values are used to generate one or more nonnegative dimensionless recognition metrics.

In some embodiments, one or more bounded recognition-weight values are derived using F_cov(r)=r/(r+X_opt), and one or more recognition-weighted molecular match outputs are generated according to R_match(C,T)=DockScore(C,T)*F_cov(D_C,T). In some embodiments, descriptor-based predictive inputs are additionally generated for the same candidate compounds and evaluated using recognition-enhanced predictive modeling. In some embodiments, one or more developability-related inputs including lipophilicity, polarity, molecular size, and metabolic-stability-related values are additionally processed using the recognition developability analysis subsystem.

In some embodiments, the resulting module outputs are combined in the composite virtual screening ranking subsystem to generate a ranked list of candidate compounds. In some embodiments, the ranked output identifies candidate compounds that maintain favorable binding-related and predictive characteristics while exhibiting stronger recognition-weighted consistency with known kinase-recognition patterns than compounds prioritized using docking-related evaluation alone. In some embodiments, one or more top-ranked compounds are selected for follow-up biochemical assay testing, selectivity evaluation, or medicinal chemistry review.

In another embodiment, the disclosed platform is used to generate recognition-enhanced predictive activity outputs for a phosphoinositide 3-kinase-related screening program. In some embodiments, a dataset of compounds having known activity measurements is used to generate processed annotation inputs, and one or more descriptor sets are calculated for each compound. In some embodiments, descriptor preparation includes missing-value handling, clipping of unstable descriptor ranges, grouping of related descriptor families, and normalization of descriptor-derived inputs.

In some embodiments, one or more nonnegative dimensionless recognition metrics are derived from one or more descriptor-related values, and corresponding bounded recognition-weight values are generated using the recognition coverage function. In some embodiments, descriptor-related predictive contributions are recognition-weighted before evaluation by a predictive model. In some embodiments, the predictive model is a wrapper model that uses an existing trained model while modifying one or more descriptor contributions through recognition weighting. In some embodiments, the predictive model is retrained using recognition-weighted features.

In some embodiments, candidate compounds associated with extreme, sparse, unstable, or weakly informative descriptor patterns are attenuated relative to otherwise comparable compounds exhibiting more recognition-consistent descriptor patterns. In some embodiments, the resulting predictive outputs are used to prioritize compounds for secondary screening, to refine a focused medicinal chemistry worklist, to support hit expansion, or to improve selection of compounds for additional profiling.

In another embodiment, the disclosed platform is used in a fragment-linking and structural optimization campaign. In some embodiments, two or more fragment candidates associated with a common target site are computationally evaluated together with one or more linker candidates or linker-bearing intermediate structures. In some embodiments, one or more conformers, fragment placements, or linked structures are processed to derive one or more interaction-related or geometric values, including pairwise distances, pharmacophore alignments, contact-related values, and one or more interaction magnitudes.

In some embodiments, one or more such values are transformed and normalized to produce one or more nonnegative dimensionless recognition metrics, and corresponding bounded recognition-weight values are generated using the recognition coverage function. In some embodiments, one or more recognition-weighted structural optimization outputs are generated according to E_opt(C)=sum over i,j of E_ij*F_cov(d_ij), where E_ij represents one or more structural or interaction-related values and d_ij represents one or more normalized structural recognition metrics.

In some embodiments, different linker candidates are ranked based on one or more resulting structural optimization outputs. In some embodiments, the disclosed workflow identifies one or more linker candidates or linked fragment arrangements exhibiting more favorable recognition-weighted structural compatibility than would be indicated by unweighted structural evaluation alone. In some embodiments, one or more such candidates are selected for further docking, synthesis planning, medicinal chemistry review, or iterative design refinement.

In another embodiment, the disclosed platform is used to triage compounds according to developability characteristics during hit-to-lead progression. In some embodiments, a set of candidate compounds already exhibiting favorable binding-related or predictive outputs is further evaluated using one or more developability-related inputs including lipophilicity, molecular weight, hydrogen-bonding-related values, permeability-related values, microsomal stability values, and toxicity-related values. In some embodiments, one or more developability-related metrics are transformed so that higher transformed values correspond to more favorable developability.

In some embodiments, one or more transformed values are normalized to generate one or more nonnegative dimensionless recognition metrics, and one or more bounded recognition-weight values are generated using the recognition coverage function. In some embodiments, a recognition-weighted developability output is generated according to M_drug_like(C)=sum over i of k_i*metric_i_norm(C), followed by S_ADMET(C) =F_cov(M_drug_like(C)). In some embodiments, compounds exhibiting otherwise favorable binding-related outputs but poor developability-related characteristics are deprioritized or filtered.

In some embodiments, compounds exhibiting balanced recognition-weighted binding-related, predictive, and developability-related characteristics are advanced for lead optimization, expanded profiling, synthesis prioritization, or additional medicinal chemistry consideration. In some embodiments, the disclosed workflow reduces the need to manually reconcile multiple disconnected developability outputs when comparing candidate compounds.

In another embodiment, the disclosed platform is used in a viral protease screening campaign. In some embodiments, a prepared viral protease structure is used together with one or more known binders, co-crystallized ligands, or interaction-pattern references relevant to the protease active site. A large candidate library is preprocessed and then evaluated using recognition molecular matching. In some embodiments, one or more predictive modeling outputs and one or more developability outputs are also generated for the same candidate compounds.

In some embodiments, the resulting subsystem outputs are harmonized and combined in the composite virtual screening ranking subsystem using one or more project-specific weights to generate a ranked list of candidate compounds for prospective testing. In some embodiments, the disclosed platform is used to identify a reduced candidate set for biochemical assay testing from a much larger initial screening set. In some embodiments, the final candidate set is selected using not only docking-related favorability but also recognition-weighted consistency with target-relevant reference features and one or more additional predictive or developability criteria.

In another embodiment, the disclosed platform is used in a G protein-coupled receptor selectivity campaign. In some embodiments, one receptor subtype is designated as a primary target and one or more related receptor subtypes are designated as secondary targets or off-targets. One or more known subtype-selective ligands and one or more subtype-relevant references are selected for the target panel. In some embodiments, one or more target-specific recognition-weighted outputs are generated for each candidate compound relative to each receptor subtype.

In some embodiments, one or more selectivity-related values are then derived using one or more ratios, differences, weighted panel comparisons, or penalized target-versus-off-target scoring functions. In some embodiments, a selectivity-related value is generated according to SelectivityScore(C)=R_match(C,Target_A)/R_match(C,Target_B), and one or more additional off-target penalties or developability filters are also applied. In some embodiments, the resulting output identifies candidate compounds exhibiting favorable recognition-weighted preference for the desired receptor subtype while avoiding one or more undesirable subtype profiles.

In some embodiments, one or more compounds prioritized using the selectivity workflow are advanced for subtype profiling, functional testing, medicinal chemistry refinement, or additional target-panel analysis. In some embodiments, the disclosed workflow can be used for subtype selectivity, target-family selectivity, anti-target avoidance, or balanced polypharmacology.

In another embodiment, the disclosed platform is used for RNA-targeted small-molecule discovery. In some embodiments, one or more structured RNA motifs or RNA-associated binding pockets are used as target inputs, and one or more known RNA-binding compounds, motif-recognition templates, pharmacophore-like representations, or structural contact patterns are used as reference inputs. In some embodiments, candidate compounds are evaluated using one or more docking-related, motif-correspondence-related, shape-related, or interaction-pattern-related values.

In some embodiments, one or more such values are processed using the shared recognition framework to generate recognition-weighted molecular matching outputs. In some embodiments, one or more predictive or developability modules are additionally applied. In some embodiments, the resulting outputs are used to prioritize candidate compounds for RNA-binding assays, motif-specific validation, selective RNA-targeting campaigns, or secondary optimization workflows. In some embodiments, the use of target-relevant RNA-recognition templates helps distinguish compounds more likely to exhibit meaningful RNA-associated interaction patterns from compounds selected using docking-related outputs alone.

In another embodiment, the disclosed platform is used in a protein-protein interaction modulation campaign. In some embodiments, one or more protein-protein interaction hot spots, contact patterns, or pharmacophore-like interface representations are used to generate one or more reference-associated recognition metrics. In some embodiments, candidate compounds are docked or otherwise positioned relative to one or more target interface regions, and one or more shape, pharmacophore, contact-pattern, or interface-recognition values are derived.

In some embodiments, one or more such values are transformed and normalized into one or more nonnegative dimensionless recognition metrics, and one or more bounded recognition-weight values are generated using the recognition coverage function. In some embodiments, one or more recognition-weighted outputs are generated to prioritize candidate compounds exhibiting favorable interface-related recognition characteristics. In some embodiments, one or more such outputs are combined with developability or predictive outputs to generate a composite prioritization output for follow-up screening, assay testing, or medicinal chemistry review.

In another embodiment, the disclosed platform is used in a covalent inhibitor discovery campaign. In some embodiments, one or more candidate compounds bearing reactive warheads are evaluated relative to a target structure containing one or more targetable residues. In some embodiments, one or more docking-related values, geometric proximity values, orientation-related values, and one or more warhead-associated or residue-associated structural values are processed to derive one or more recognition metrics.

In some embodiments, one or more recognition-weighted molecular matching outputs and one or more recognition-weighted structural optimization outputs are generated and combined with one or more developability-related outputs. In some embodiments, the resulting workflow is used to prioritize compounds exhibiting favorable recognition-weighted proximity and orientation characteristics relative to the targetable residue while maintaining acceptable developability characteristics. In some embodiments, one or more such compounds are selected for covalent binding assays, selectivity testing, or analog design.

In another embodiment, the disclosed platform is used in connection with a generative molecular design workflow. In some embodiments, a generative model produces one or more proposed candidate compounds, and the proposed compounds are routed through one or more modules of the disclosed platform to generate recognition-weighted outputs. In some embodiments, one or more recognition-weighted molecular matching outputs, one or more recognition-enhanced predictive outputs, one or more recognition-weighted structural optimization outputs, and one or more recognition-weighted developability outputs are combined to produce one or more composite prioritization or reward values.

In some embodiments, one or more such values are used to guide subsequent rounds of molecular generation, candidate refinement, scaffold exploration, analog enumeration, or medicinal chemistry suggestion generation. In some embodiments, the disclosed platform thereby provides a recognition-weighted reward or scoring layer for iterative computational molecular design. In some embodiments, this can support generation of candidate compounds more closely aligned with target-relevant recognition characteristics and project-specific prioritization objectives.

In some embodiments, one or more of the foregoing examples are performed prospectively, retrospectively, iteratively, or in hybrid form. In some embodiments, one or more examples are performed using a single target, multiple targets, a target family, one or more off-target panels, or one or more project-stage-specific workflows. In some embodiments, one or more examples are combined within a larger end-to-end drug discovery campaign.

In some embodiments, one or more examples can use a single reference ligand, multiple reference ligands, one or more pharmacophore models, one or more interaction fingerprints, one or more shape or electrostatic models, or combinations thereof. In some embodiments, one or more examples can use fixed weights, project-specific weights, target-specific weights, or adaptively updated weights.

In some embodiments, one or more examples can use experimentally measured target structures, predicted target structures, homology-derived target structures, or combinations thereof. In some embodiments, one or more examples can use public datasets, private enterprise datasets, literature-curated datasets, assay-derived datasets, or combinations thereof.

In some embodiments, the disclosed examples demonstrate that the platform can be applied across diverse drug discovery settings, including docking-focused screening, predictive modeling, structural optimization, developability triage, selectivity analysis, RNA-targeting, protein-protein interaction modulation, covalent discovery, and generative design. In some embodiments, use of the shared recognition framework across such settings enables a unified, bounded, and configurable computational basis for identifying, ranking, filtering, selecting, and optimizing candidate compounds.

In some embodiments, the disclosed platform can reduce reliance on ad hoc manual integration of disconnected computational outputs by providing a common recognition-weighted framework across multiple modules. In some embodiments, the disclosed platform can support more consistent comparison of candidate compounds across distinct discovery stages, target classes, and campaign objectives.

In some embodiments, the disclosed platform can be configured to emphasize hit identification, hit expansion, lead optimization, selectivity engineering, off-target risk reduction, developability triage, scaffold prioritization, or combinations thereof. In some embodiments, different subsystems can be emphasized, omitted, or combined differently depending on project stage and available data.

In some embodiments, the disclosed platform can operate using one or more stand-alone subsystems or as a coordinated multi-module platform. In some embodiments, one or more outputs from one campaign can be reused as inputs for another campaign, including use of updated references, updated thresholds, updated weights, updated models, or updated target-panel settings.

In some embodiments, the disclosed workflows can be implemented locally, remotely, in a distributed computing environment, in a cloud-based environment, or in combinations thereof. In some embodiments, one or more outputs can be stored, versioned, logged, exported, displayed, or reused in subsequent analyses, validation exercises, or iterative design cycles.

The foregoing examples are illustrative of the flexibility and breadth of the disclosed recognition-optimized drug discovery platform and are not intended to limit the scope of the claims. A person of ordinary skill in the art, having the benefit of this disclosure, will appreciate that additional targets, scoring configurations, molecular classes, workflow variants, and implementation arrangements can be used without departing from the spirit and scope of the invention.

The disclosed platform accordingly provides a recognition-weighted computational framework that can be used across multiple drug discovery contexts to transform heterogeneous inputs into bounded, interpretable, and campaign-configurable outputs for identifying, ranking, filtering, selecting, and optimizing candidate compounds.

In some embodiments, the disclosed platform can be implemented using one or more modules that operate sequentially, iteratively, concurrently, or independently. In some embodiments, one or more modules can be omitted where a given project does not require the corresponding functionality. In some embodiments, one or more additional modules can be included to process target-class-specific inputs, campaign-specific metrics, or project-specific decision criteria while still using the shared recognition framework described herein.

In some embodiments, one or more recognition metrics used by the disclosed platform are derived from transformed raw values that are normalized relative to one or more selected scales, reference values, percentile-derived values, target-specific normalization factors, project-specific normalization factors, or combinations thereof. In some embodiments, a raw value can be transformed by negation, offsetting, clipping, absolute-value conversion, reciprocal conversion, exponential conversion, threshold conversion, rank-based conversion, percentile-based conversion, target-window conversion, or other monotonic transformation before generation of a nonnegative dimensionless recognition metric.

In some embodiments, the disclosed platform can use a single reference-associated recognition metric for a given compound-target pair. In some embodiments, multiple reference-associated recognition metrics can be generated and combined using one or more maximum functions, average functions, weighted combinations, consensus procedures, grouped combinations, or other derived values. In some embodiments, different reference strategies can be used for different targets, different target families, different project stages, or different workflows within the same platform.

In some embodiments, one or more subsystem outputs can be combined using weighted sums, weighted averages, geometric means, harmonic means, rank fusion procedures, gated selection procedures, hierarchical aggregation procedures, Pareto-based prioritization procedures, or combinations thereof. In some embodiments, the disclosed platform can generate one ranked list, multiple ranked lists, one filtered worklist, multiple filtered worklists, one deprioritized set, one selectivity panel output, or combinations thereof depending on project objectives.

In some embodiments, the disclosed platform can be used with small molecules, fragments, macrocycles, peptidomimetics, covalent compounds, payload candidates, nucleic-acid binders, protein-protein interaction modulators, or other candidate structures. In some embodiments, the disclosed platform can be used with proteins, nucleic acids, protein complexes, receptor families, ion channels, transporters, enzymes, viral targets, bacterial targets, fungal targets, inflammatory targets, oncology targets, rare-disease targets, or other biological targets.

In some embodiments, one or more outputs of the disclosed platform can be used for hit identification, hit expansion, lead optimization, selectivity optimization, off-target avoidance, safety triage, developability triage, scaffold prioritization, fragment elaboration, macrocycle assessment, analog design, assay planning, medicinal chemistry planning, or other discovery decisions. In some embodiments, one or more outputs can also be used to update references, thresholds, weights, predictive models, workflow policies, or target-panel definitions for subsequent screening rounds.

In some embodiments, the disclosed platform can improve consistency across multiple computational drug discovery stages by using the shared recognition coverage function across otherwise heterogeneous workflows. In some embodiments, the disclosed platform can reduce reliance on disconnected scoring systems and ad hoc manual reconciliation by providing a common bounded recognition-weighted framework for molecular matching, predictive modeling, structural optimization, developability analysis, and composite prioritization.

In some embodiments, the disclosed platform can attenuate weakly informative, structurally inconsistent, unstable, or poorly calibrated contributions without requiring that all workflows use the same underlying raw inputs or the same underlying software tools. In some embodiments, this can improve practical integration of docking-related values, descriptor-based predictions, structural optimization terms, developability metrics, target-panel comparisons, and campaign-level prioritization criteria.

It will be understood that the particular arrangements, modules, workflows, equations, transformations, and examples described herein are illustrative and not limiting. A person of ordinary skill in the art, having the benefit of this disclosure, will appreciate that various substitutions, modifications, rearrangements, additions, omissions, and combinations can be made without departing from the spirit and scope of the invention as defined by the claims.

Accordingly, the present disclosure provides computational methods, systems, and non-transitory computer-readable media for recognition-optimized drug discovery in which one or more heterogeneous inputs are transformed into one or more nonnegative dimensionless recognition metrics, processed using a bounded recognition coverage function, and used to generate one or more recognition-weighted outputs for identifying, ranking, filtering, selecting, deprioritizing, or optimizing candidate compounds across one or more drug discovery workflows.

What is claimed is:

1. A computer-implemented method for evaluating candidate compounds for a biological target, the method comprising:
receiving, by one or more processors, compound data for a candidate compound, target data for the biological target, and reference data associated with at least one reference representation relevant to the biological target;
generating, by the one or more processors, a docking-related value representing a predicted interaction of the candidate compound with the biological target;
generating, by the one or more processors, a similarity-related value representing structural or interaction-related correspondence between the candidate compound and the at least one reference representation;
transforming and normalizing the similarity-related value to obtain a nonnegative dimensionless recognition metric;
processing the nonnegative dimensionless recognition metric with a recognition coverage function given by F_cov(r)=r/(r+X_opt) to generate a bounded recognition-weight value, wherein r is the nonnegative dimensionless recognition metric and X_opt is a constant;
combining the docking-related value with the bounded recognition-weight value to generate a recognition-weighted molecular match score for the candidate compound relative to the biological target; and
selecting the candidate compound for a follow-on action when (i) the recognition-weighted molecular match score exceeds a threshold stored in memory or (ii) the recognition-weighted molecular match score is within a top-K ranked set for the biological target, and outputting, by the one or more processors, data indicating the recognition-weighted molecular match score and the selecting for use in ranking, filtering, deprioritizing, or optimizing the candidate compound.

2. The method of claim 1, wherein generating the similarity-related value comprises generating at least one of a fingerprint similarity value, a pharmacophore correspondence value, a shape similarity value, an electrostatic similarity value, an interaction-pattern similarity value, a graph-based similarity value, or an RMSD-derived value.

3. The method of claim 1, wherein transforming and normalizing the similarity-related value comprises generating a similarity value Sim in a range of 0 to 1, dividing Sim by a selected normalizing scale S_ref to obtain an intermediate value, and clipping the intermediate value to obtain the nonnegative dimensionless recognition metric r according to r=clip(Sim/S_ref, 0, r_max), wherein r_max is selected in a range of 1 to 10.

4. The method of claim 1, wherein the recognition coverage function generates the bounded recognition-weight value such that the bounded recognition-weight value is zero when the nonnegative dimensionless recognition metric is zero, increases as the nonnegative dimensionless recognition metric increases, and remains less than one for finite positive values of the nonnegative dimensionless recognition metric.

5. The method of claim 1, wherein the docking-related value is transformed prior to combining such that a transformed docking value is computed as DockScore(C,T)=clip(−E_dock(C,T)+b, 0, S_max), where E_dock(C,T) is a docking energy, b is an offset selected in a range of 0 to 20, and S_max is selected in a range of 10 to 50, and wherein the recognition-weighted molecular match score is generated using the transformed docking value as the docking-related value.

6. The method of claim 1, wherein transforming and normalizing the similarity-related value comprises selecting a scale for the normalizing based on at least one of (i) a percentile-derived value computed over similarity-related values for a compound library associated with the biological target or (ii) a reference-derived value computed from the at least one reference representation.

7. The method of claim 1, wherein generating the similarity-related value comprises generating respective similarity-related values relative to a plurality of reference representations and aggregating the respective similarity-related values using at least one of a maximum, a median, or a weighted combination prior to transforming and normalizing.

8. A system for computational drug discovery, comprising:
one or more processors; and
memory storing instructions that, when executed by the one or more processors, cause the system to:
receive compound data, target data, reference data, descriptor data, and developability-related data;
preprocess the compound data, the target data, the reference data, the descriptor data, and the developability-related data to generate processed inputs;
generate, from the processed inputs, a recognition-weighted molecular match output, a recognition-enhanced predictive output, and a recognition-weighted developability output, including by deriving from at least a portion of the processed inputs a plurality of nonnegative dimensionless recognition metrics and processing the plurality of nonnegative dimensionless recognition metrics with a recognition coverage function given by F_cov(r)=r/(r+X_opt) to generate a plurality of bounded recognition-weight values;
harmonize at least two outputs selected from the recognition-weighted molecular match output, the recognition-enhanced predictive output, and the recognition-weighted developability output using a deterministic scale-harmonization rule comprising at least one of percentile conversion, z-score normalization, or min-max scaling; and
combine the at least two harmonized outputs using an aggregation operation comprising at least one of a weighted sum, a weighted average, a geometric mean, a rank-fusion operation, or a minimum/maximum operator to generate a composite prioritization output for a candidate compound, wherein weighting coefficients used in the aggregation operation are nonnegative and sum to 1.

9. The system of claim 8, wherein the instructions further cause the system to generate the recognition-weighted molecular match output by combining a transformed docking-related value with a bounded recognition-weight value derived from a similarity-related value associated with at least one reference ligand, pharmacophore representation, interaction fingerprint, shape model, or electrostatic model.

10. The system of claim 8, wherein the instructions further cause the system to generate the recognition-enhanced predictive output by applying the bounded recognition coverage function to one or more descriptor values, grouped descriptor values, latent variables, embeddings, or feature-family aggregates associated with the candidate compound.

11. The system of claim 8, wherein the instructions further cause the system to generate the recognition-weighted developability output by transforming one or more physicochemical metrics or absorption-distribution-metabolism-excretion-toxicity-related metrics so that larger transformed values correspond to more favorable developability and then processing one or more normalized transformed values with the bounded recognition coverage function.

12. The system of claim 8, wherein the instructions further cause the system to store, in at least one data store, at least one of (i) a selected normalization scale, (ii) the threshold or the K value, (iii) X_opt, or (iv) a timestamp, in association with generating at least one ranked, filtered, selected, deprioritized, or optimized output for the candidate compound.

13. The system of claim 8, wherein the instructions further cause the system to evaluate the candidate compound relative to a plurality of biological targets, generate target-specific recognition-weighted outputs for the plurality of biological targets, and generate a selectivity-related output based on a comparison of the target-specific recognition-weighted outputs.

14. The system of claim 8, wherein the instructions further cause the system to update at least one of a parameter, a threshold, a reference set, or a model based on assay-derived results or medicinal chemistry review inputs associated with one or more previously evaluated candidate compounds.

15. A non-transitory computer-readable medium storing instructions that, when executed by one or more processors, cause the one or more processors to:
receive data associated with a candidate compound, a biological target, and at least one target-relevant reference;
generate a first transformed value associated with a predicted interaction of the candidate compound with the biological target;
generate a second transformed value associated with structural, pharmacophoric, shape-based, electrostatic, graph-based, or interaction-pattern correspondence between the candidate compound and the at least one target-relevant reference;
normalize the second transformed value to obtain a nonnegative dimensionless recognition metric;
process the nonnegative dimensionless recognition metric using a recognition coverage function given by F_cov(r)=r/(r+X_opt) to generate a bounded recognition-weight value, wherein r is the nonnegative dimensionless recognition metric and X_opt is a constant;
combine the first transformed value with the bounded recognition-weight value to generate a recognition-weighted score for the candidate compound; and
select the candidate compound for a follow-on action when (i) the recognition-weighted score exceeds a threshold stored in memory or (ii) the recognition-weighted score is within a top-K ranked set for the biological target, and store or output data indicating the recognition-weighted score and the selecting.

16. The non-transitory computer-readable medium of claim 15, wherein the instructions further cause the one or more processors to generate the first transformed value by converting a docking energy, affinity estimate, interaction score, pose-quality value, or docking rank into a value for which larger values correspond to more favorable predicted interaction.

17. The non-transitory computer-readable medium of claim 15, wherein X_opt=phi/pi.

18. The non-transitory computer-readable medium of claim 15, wherein the instructions further cause the one or more processors to generate a recognition-enhanced predictive output for the candidate compound by processing one or more descriptor-derived inputs with the bounded recognition coverage function and evaluating a predictive model using one or more resulting weighted predictive inputs.

19. The non-transitory computer-readable medium of claim 15, wherein the instructions further cause the one or more processors to generate a recognition-weighted developability output for the candidate compound by processing one or more transformed and normalized developability-related inputs with the bounded recognition coverage function.

20. The non-transitory computer-readable medium of claim 15, wherein the instructions further cause the one or more processors to combine the recognition-weighted score with at least one additional output selected from a recognition-enhanced predictive output, a recognition-weighted structural optimization output, or a recognition-weighted developability output to generate a composite prioritization output for the candidate compound.
